(19) United States
(12) Patent Application Publication
(10) Pub. No.: US 2003/0017978 A1
(43) Pub. Date: Jan. 23, 2003

(54) Spf1-related transcription factors

(76) Inventors: Qun Zhu, West Chester, PA (US); Omolayo O Famodu, Newark, DE (US)

Correspondence Address: Thomas M Rizzo, E I Du Pont De Nemours And Company, Legal Patent, Wilmington, DE, 19898, US

(21) Appl. No.: 09/890,811

(22) PCT Filed: Dec. 22, 2000
(86) PCT No.: PCT/US00/35310

(51) Int. Cl. A61K 38/17; C07H 21/04; C12N 9/22; C12P 21/02; C12N 5/06

(52) U.S. Cl. 514/12; 435/199; 435/69.1; 435/320.1; 435/325; 536/23.2

(57) ABSTRACT

This invention relates to an isolated nucleic acid fragment encoding an SPF1-related transcription factor. The invention also relates to the construction of a chimeric gene encoding all or a portion of the SPF1-related transcription factor, in sense or antisense orientation, wherein expression of the chimeric gene results in production of altered levels of the SPF1-related transcription factor in a transformed host cell.

This application claims the benefit of U.S. Provisional Application No. 60/174,325, filed Jan. 4, 2000.

### FIELD OF THE INVENTION

This invention is in the field of plant molecular biology. More specifically, this invention pertains to nucleic acid fragments encoding SPF1-related transcription factors in plants and seeds.

### BACKGROUND OF THE INVENTION

Disease resistance is mediated by an array of defense responses that are coordinately regulated. In addition to preexisting defense structures such as waxes on leaf and fruit surfaces, thick cuticle, and thick and tough outer walls of epidermal cells, defense strategies include formation of histological and cellular defense structures in response to infection which include cork layers, abscission layers, tyloses, gums, callose papillae, and the hypersensitive response. Biochemical defense responses include lignification of cell walls, production of toxic metabolites like phenolic compounds and phytoalexins, and induction of particular enzymes like those involved in degrading the invading pathogen and phytoalexin biosynthesis, and phenol-oxidizing enzymes. Understandably, disease resistance mechanisms vary from one pathosystem to another, in terms of the defense strategies involved and their timing.

Defense responses have been intensively studied in suspension-cultured parsley (Petroselinum crispum) cells using a defined oligopeptide elicitor molecule (Pep25) which initiates several defense responses observed in whole-plant tissue infections (Nürnberger et al. (1994) Cell 78:449-460; Hahlbrock et al. (1995) Proc Natl Acad Sci USA 92:4150-4157) including large and transient increases in several ion fluxes, H₂O₂ formation, phosphorylation of various proteins (Dietrich et al. (1990) J Biol Chem 265:6360-6368), and activation of several defense-related genes (Somssich et al. (1989) Plant Mol Biol 12:227-234). Among the proteins whose synthesis is induced is PR1, a pathogenesis-related protein that is encoded by a family of three genes, PR1-1, PR1-2 and PR1-3. Implicated in PR1 gene transcriptional activation are the transcription factors WRKY1, 2 and 3 (Rushton et al. (1996) EMBO J 15:5690-5700; Eulgem et al. (1999) EMBO J 18:4689-4699). WRKY1, 2, and 3 have been found via South-Western screening to bind fungal elicitor responsive elements in the PR1-1 and PR1-2 promoters (Rushton et al. (1996) EMBO J 15:5690-5700). WRKY1 and WRKY3 mRNA levels showed a transient and extremely rapid increase while WRKY2 mRNA level showed a concomitant transient decrease, upon elicitor (Pep25) induction (Rushton et al. (1996) EMBO J 15:5690-5700), suggesting that WRKY 1, 2 and 3 play a key role in PR1 gene activation.

WRKY proteins have been identified in a variety of plant species and appear to be plant-specific. They all have one or two copies of the highly conserved WRKY domain which consists of a novel type of zinc finger motif (C-X₄₋₅-C-X₂₂₋₂₃-H-X-H) at the C-terminus, and the N-terminal sequence WRKYGQK (hence the name). Outside the WRKY domain, the similarity among member proteins of the WRKY family is considerably lower, although like other transcription factors, they have putative transcriptional activation domains and nuclear localization signals. In addition to defense gene regulation, WRKY proteins have also been implicated to play a role in hormonal regulation (Rushton et al. (1995) Plant Mol Biol 29:691-702) and carbohydrate regulation (Ishiguro and Nakamura (1994) Mol Gen Genet 244:563-571). It is apparent that WRKY proteins play a key role in transcriptional activation of key genes in diverse plant processes.

Related to WRKY proteins is the SPF1 DNA-binding protein, which binds to the SP8a and SP8b sequences present in the 5′ upstream regions of genes that encode sporamin and beta-amylase, two major proteins in tuberous roots of sweet potato (Ishiguro and Nakamura (1994) Mol Gen Genet 244:563-571) suggesting that SPF1 maybe involved in carbohydrate regulation and storage protein accumulation. A cDNA encoding a putative SPF1-type DNA-binding protein has also been isolated from cucumber with an expression level that increases in cotyledons as they expand and become photosynthetic and remains high in senescence (Kim et al. (1997) Gene 185:265-269). More recently, the TTG2 gene of Arabidopsis that regulates trichome development and the production of pigment and mucilage in seed coats was found to encode a transcription factor with two SPF1 zinc finger-like domains, suggesting that SPF1 family of transcription factors is involved in a diverse array of plant processes (Johnson and Smyth, 9^{th} International Conference on Arabidopsis Research, Jun. 24-29, 1998).

There is a great deal of interest in isolating genes that encode SPF1 homolog proteins involved in transcriptional activation of various genes in plants. These genes may be used in plants to control transcription of particular genes, chimeric or otherwise, during plant growth, development and response to environmental cues. Accordingly, the availability of nucleic acid sequences encoding all or a portion of SPF1 homolog proteins would facilitate studies to better understand the mechanism of transcriptional activation in plants and promoter specificity of the different SPF1 proteins, and could provide genetic tools to enhance or otherwise alter the level of accumulation of seed protein in plants as well as other processes regulated by the SPF1 family of transcription factors.

### SUMMARY OF THE INVENTION

The present invention concerns an isolated polynucleotide comprising: (a) a first nucleotide sequence encoding a first polypeptide comprising at least 100 amino acids, wherein the amino acid sequence of the first polypeptide and the amino acid sequence of SEQ ID NO:2 or SEQ ID NO:4 have at least 70%, 80%, 85%, 90%, or 95% identity based on the Clustal alignment method, (b) a second nucleotide sequence encoding a second polypeptide comprising at least 150 amino acids, wherein the amino acid sequence of the second polypeptide and the amino acid sequence of SEQ ID NO:12 have at least 70%, 80%, 85%, 90%, or 95% identity based on the Clustal alignment method, (c) a third nucleotide sequence encoding a third polypeptide comprising at least 250 amino acids, wherein the amino acid sequence of the third polypeptide and the amino acid sequence of SEQ ID NO:6 or SEQ ID NO:8 have at least 70%, 80%, 85%, 90%, or 95% identity based on the Clustal alignment method, (d) a fourth nucleotide sequence encoding a fourth polypeptide comprising at least 300 amino acids, wherein the amino acid sequence of the fourth polypeptide and the amino acid sequence of SEQ ID NO:10 have at least 80%, 85%, 90%, or 95% identity based on the Clustal alignment method, or (e) the complement of the first, second, third, or fourth nucleotide sequence, wherein the complement and the first, second, third, or fourth nucleotide sequence contain the same number of nucleotides and are 100% complementary. The first polypeptide preferably comprises the amino acid sequence of SEQ ID NO:2 or SEQ ID NO:4, the second polypeptide preferably comprises the amino acid sequence of SEQ ID NO:12, the third polypeptide preferably comprises the amino acid sequence of SEQ ID NO:6 or SEQ ID NO:8, and the fourth polypeptide preferably comprises the amino acid sequence of SEQ ID NO:10. The first nucleotide sequence preferably comprises the nucleotide sequence of SEQ ID NO:1 or SEQ ID NO:3, the second nucleotide sequence preferably comprises the nucleotide sequence of SEQ ID NO:11, the third nucleotide sequence preferably comprises the nucleotide sequence of SEQ ID NO:5 or SEQ ID NO:7, and the fourth nucleotide sequence preferably comprises the nucleotide sequence of SEQ ID NO:9. The first, second, third, and fourth polypeptides preferably are SPF1-related transcription factors.

In a second embodiment, the present invention relates to a chimeric gene comprising any of the isolated polynucleotides of the present invention operably linked to a regulatory sequence, and a cell, a plant, and a seed comprising the chimeric gene.

In a third embodiment, the present invention relates to a vector comprising any of the isolated polynucleotides of the present invention.

In a fourth embodiment, the present invention relates to an isolated polynucleotide fragment comprising a nucleotide sequence comprised by any of the polynucleotides of the present invention, wherein the nucleotide sequence contains at least 30, 40, or 60 nucleotides.

In a fifth embodiment, the present invention concerns an isolated polypeptide comprising: (a) a first amino acid sequence comprising at least 100 amino acids, wherein the first amino acid sequence and the amino acid sequence of SEQ ID NO:2 or SEQ ID NO:4 have at least 70%, 80%, 85%, 90%, or 95% identity based on the Clustal alignment method, (b) a second amino acid sequence comprising at least 150 amino acids, wherein the second amino acid sequence and the amino acid sequence of SEQ ID NO:12 have at least 70%, 80%, 85%, 90%, or 95% identity based on the Clustal alignment method, (c) a third amino acid sequence comprising at least 250 amino acids, wherein the third amino acid sequence and the amino acid sequence of SEQ ID NO:6 or SEQ ID NO:8 have at least 70%, 80%, 85%, 90%, or 95% identity based on the Clustal alignment method, or (d) a fourth amino acid sequence comprising at least 300 amino acids, wherein the fourth amino acid sequence and the amino acid sequence of SEQ ID NO:10 have at least 80%, 85%, 90%, or 95% identity based on the Clustal alignment method. The first amino acid sequence preferably comprises the amino acid sequence of SEQ ID NO:2 or SEQ ID NO:4, the second amino acid sequence preferably comprises the amino acid sequence of SEQ ID NO:12, the third amino acid sequence preferably comprises the amino acid sequence of SEQ ID NO:6 or SEQ ID NO:8, and the fourth amino acid sequence preferably comprises the amino acid sequence of SEQ ID NO:10. The polypeptide preferably is an SPF1-related transcription factor.

In a sixth embodiment, the present invention relates to a method for transforming a cell comprising transforming a cell with any of the isolated polynucleotides of the present invention, and the cell transformed by this method. Advantageously, the cell is eukaryotic, e.g., a yeast or plant cell, or prokaryotic, e.g., a bacterium.

In a seventh embodiment, the present invention relates to a method for producing a transgenic plant comprising transforming a plant cell with any of the isolated polynucleotides of the present invention and regenerating a plant from the transformed plant cell, the transgenic plant produced by this method, and the seed obtained from this transgenic plant.

In an eighth embodiment, the present invention relates to a virus, preferably a baculovirus, comprising any of the isolated polynucleotides of the present invention or any of the chimeric genes of the present invention.

In a ninth embodiment, the invention relates to a method of selecting an isolated polynucleotide that affects the level of expression of an SPF1-related transcription factor polypeptide or enzyme activity in a host cell, preferably a plant cell, the method comprising the steps of: (a) constructing an isolated polynucleotide of the present invention or an isolated chimeric gene of the present invention; (b) introducing the isolated polynucleotide or the isolated chimeric gene into a host cell; (c) measuring the level of the SPF1-related transcription factor polypeptide or enzyme activity in the host cell containing the isolated polynucleotide; and (d) comparing the level of the SPF1-related transcription factor polypeptide or enzyme activity in the host cell containing the isolated polynucleotide with the level of the SPF1-related transcription factor polypeptide or enzyme activity in the host cell that does not contain the isolated polynucleotide.

In a tenth embodiment, the invention concerns a method of obtaining a nucleic acid fragment encoding a substantial portion of an SPF1-related transcription factor polypeptide, preferably a plant SPF1-related transcription factor polypeptide, comprising the steps of: synthesizing an oligonucleotide primer comprising a nucleotide sequence of at least one of 60 (preferably at least one of 40, most preferably at least one of 30) contiguous nucleotides derived from a nucleotide sequence selected from the group consisting of SEQ ID NOs:1, 3, 5, 7, 9, and 11, and the complement of such nucleotide sequences; and amplifying a nucleic acid fragment (preferably a cDNA inserted in a cloning vector) using the oligonucleotide primer. The amplified nucleic acid fragment preferably will encode a substantial portion of an SPF1-related transcription factor polypeptide amino acid sequence.

In an eleventh embodiment, this invention relates to a method of obtaining a nucleic acid fragment encoding all or a substantial portion of the amino acid sequence encoding an SPF1-related transcription factor polypeptide comprising the steps of: probing a cDNA or genomic library with an isolated polynucleotide of the present invention; identifying a DNA clone that hybridizes with an isolated polynucleotide of the present invention; isolating the identified DNA clone; and sequencing the cDNA or genomic fragment that comprises the isolated DNA clone.

In a twelfth embodiment, this invention concerns a method for positive selection of a transformed cell comprising: (a) transforming a host cell with the chimeric gene of the present invention or an expression cassette of the present invention; and (b) growing the transformed host cell, preferably a plant cell, such as a monocot or a dicot, under conditions which allow expression of the SPF1-related transcription factor polynucleotide in an amount sufficient to complement a null mutant to provide a positive selection means.

In a thirteenth embodiment, this invention relates to a method of altering the level of expression of an SPF1-related transcription factor in a host cell comprising: (a) transforming a host cell with a chimeric gene of the present invention; and (b) growing the transformed host cell under conditions that are suitable for expression of the chimeric gene wherein expression of the chimeric gene results in production of altered levels of the SPF1-related transcription factor in the transformed host cell.

### BRIEF DESCRIPTION OF THE DRAWING AND SEQUENCE LISTINGS

The invention can be more fully understood from the following detailed description and the accompanying drawing and Sequence Listing which form a part of this application.

FIG. 1 depicts the amino acid sequence alignment between the SPF1-related transcription factors encoded by the nucleotide sequences derived from rice clone rlr24.pk0007.a8 (SEQ ID NO:6), rice clone rlr24.pk0069.h10 (SEQ ID NO:8), and soybean clone slslc.pk033.c17 (SEQ ID NO:10), and the SPF1 transcription factor encoded by a cDNA isolated from Ipomoea batatas (NCBI GenBank Identifier (GI) No. 1076685) (SEQ ID NO:13). Amino acids which are conserved among all and at least two sequences with an amino acid at that position are indicated with an asterisk (*). Dashes are used by the program to maximize alignment of the sequences.

Table 1 lists the polypeptides that are described herein, the designation of the cDNA clones that comprise the nucleic acid fragments encoding polypeptides representing all or a substantial portion of these polypeptides, and the corresponding identifier (SEQ ID NO:) as used in the attached Sequence Listing. Table 1 also identifies the cDNA clones as individual ESTs (“EST”), the sequences of the entire cDNA inserts comprising the indicated cDNA clones (“FIS”), contigs assembled from two or more ESTs (“Contig”), contigs assembled from an FIS and one or more ESTs or PCR fragment sequence (“Contig*”), or sequences encoding the entire protein derived from an FIS, a contig, or an FIS and PCR fragment sequence (“CGS”). SEQ ID NOs: presented herein correspond to SEQ ID NOs: presented in U.S. Provisional Application No. 60/174,325, filed Jan. 4, 2000. The sequence descriptions and Sequence Listing attached hereto comply with the rules governing nucleotide and/or amino acid sequence disclosures in patent applications as set forth in 37 C.F.R. §1.821-1.825.

TABLE 1
SPF1-Related Transcription Factors

| | | | SEQ ID NO: | |
|---|---|---|---|---|
| Protein (Plant Source) | Clone Designation | Status | (Nucleotide) | (Amino Acid) |
| SPF 1-Related Transcription | ci11c.pk001.e13 | EST | 1 | 2 |
| Factor (Corn) | | | | |
| SPF1-Related Transcription | p0128.cpiar39r | EST | 3 | 4 |
| Factor (Corn) | | | | |
| SPF1-Related Transcription | r1r24.pk0007.a8 | CGS | 5 | 6 |
| Factor (Rice) | (FIS) | | | |
| SPF1-Related Transcription | r1r24.pk0069.h10 | CGS | 7 | 8 |
| Factor (Rice) | (FIS) | | | |
| SPF1-Related Transcription | s1s1c.pk033.c17 | CGS | 9 | 10 |
| Factor (Soybean) | (FIS) | | | |
| SPF1-Related Transcription | w1mk1.pk0035.d9 | FIS | 11 | 12 |
| Factor (Wheat) | | | | |

SEQ ID NO:13 sets for the amino acid sequence of SPF1 transcription factor encoded by a cDNA isolated from Ipomoea batatas (NCBI GI No. 1076685).

SEQ ID NO:14 is the sequence of an oligonucleotide used to create a BamHI site in clone rlr24.pk0007.a8. Details are set forth in Example 7.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R §1.822.

### DETAILED DESCRIPTION OF THE INVENTION

In the context of this disclosure, a number of terms shall be utilized. The terms “polynucleotide”, “polynucleotide sequence”, “nucleic acid sequence”, and “nucleic acid fragment”/“isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. An isolated polynucleotide of the present invention may include at least one of 60 contiguous nucleotides, preferably at least one of 40 contiguous nucleotides, most preferably one of at least 30 contiguous nucleotides derived from SEQ ID NOs:1, 3, 5, 7, 9, or 11, or the complement of such sequences.

The term “isolated” polynucleotide refers to a polynucleotide that is substantially free from other nucleic acid sequences, such as other chromosomal and extrachromosomal DNA and RNA, that normally accompany or interact with it as found in its naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

The term “recombinant” means, for example, that a nucleic acid sequence is made by an artificial combination of two otherwise separated segments of sequence, e.g., by chemical synthesis or by the manipulation of isolated nucleic acids by genetic engineering techniques.

As used herein, “contig” refers to a nucleotide sequence that is assembled from two or more constituent nucleotide sequences that share common or overlapping regions of sequence homology. For example, the nucleotide sequences of two or more nucleic acid fragments can be compared and aligned in order to identify common or overlapping sequences. Where common or overlapping sequences exist between two or more nucleic acid fragments, the sequences (and thus their corresponding nucleic acid fragments) can be assembled into a single contiguous nucleotide sequence.

As used herein, “substantially similar” refers to nucleic acid fragments wherein changes in one or more nucleotide bases results in substitution of one or more amino acids, but do not affect the functional properties of the polypeptide encoded by the nucleotide sequence. “Substantially similar” also refers to nucleic acid fragments wherein changes in one or more nucleotide bases does not affect the ability of the nucleic acid fragment to mediate alteration of gene expression by gene silencing through for example antisense or co-suppression technology. “Substantially similar” also refers to modifications of the nucleic acid fragments of the instant invention such as deletion or insertion of one or more nucleotides that do not substantially affect the functional properties of the resulting transcript vis-à-vis the ability to mediate gene silencing or alteration of the functional properties of the resulting protein molecule. It is therefore understood that the invention encompasses more than the specific exemplary nucleotide or amino acid sequences and includes functional equivalents thereof. The terms “substantially similar” and “corresponding substantially” are used interchangeably herein.

Substantially similar nucleic acid fragments may be selected by screening nucleic acid fragments representing subfragments or modifications of the nucleic acid fragments of the instant invention, wherein one or more nucleotides are substituted, deleted and/or inserted, for their ability to affect the level of the polypeptide encoded by the unmodified nucleic acid fragment in a plant or plant cell. For example, a substantially similar nucleic acid fragment representing at least one of 30 contiguous nucleotides derived from the instant nucleic acid fragment can be constructed and introduced into a plant or plant cell. The level of the polypeptide encoded by the unmodified nucleic acid fragment present in a plant or plant cell exposed to the substantially similar nucleic fragment can then be compared to the level of the polypeptide in a plant or plant cell that is not exposed to the substantially similar nucleic acid fragment.

For example, it is well known in the art that antisense suppression and co-suppression of gene expression may be accomplished using nucleic acid fragments representing less than the entire coding region of a gene, and by using nucleic acid fragments that do not share 100% sequence identity with the gene to be suppressed. Moreover, alterations in a nucleic acid fragment which result in the production of a chemically equivalent amino acid at a given site, but do not effect the functional properties of the encoded polypeptide, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products. Consequently, an isolated polynucleotide comprising a nucleotide sequence of at least one of 60 (preferably at least one of 40, most preferably at least one of 30) contiguous nucleotides derived from a nucleotide sequence selected from the group consisting of SEQ ID NOs:1, 3, 5, 7, 9, and 11, and the complement of such nucleotide sequences may be used in methods of selecting an isolated polynucleotide that affects the expression of an SPF1-related transcription factor polypeptide in a host cell. A method of selecting an isolated polynucleotide that affects the level of expression of a polypeptide in a virus or in a host cell (eukaryotic, such as plant or yeast, prokaryotic such as bacterial) may comprise the steps of: constructing an isolated polynucleotide of the present invention or an isolated chimeric gene of the present invention; introducing the isolated polynucleotide or the isolated chimeric gene into a host cell; measuring the level of a polypeptide or enzyme activity in the host cell containing the isolated polynucleotide; and comparing the level of a polypeptide or enzyme activity in the host cell containing the isolated polynucleotide with the level of a polypeptide or enzyme activity in a host cell that does not contain the isolated polynucleotide.

Moreover, substantially similar nucleic acid fragments may also be characterized by their ability to hybridize. Estimates of such homology are provided by either DNA-DNA or DNA-RNA hybridization under conditions of stringency as is well understood by those skilled in the art (Hames and Higgins, Eds. (1985) Nucleic Acid Hybridisation, IRL Press, Oxford, U.K.). Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6× SSC, 0.5% SDS at room temperature for 15 min, then repeated with 2× SSC, 0.5% SDS at 45° C. for 30 min, and then repeated twice with 0.2× SSC, 0.5% SDS at 50° C. for 30 min. A more preferred set of stringent conditions uses higher temperatures in which the washes are identical to those above except for the temperature of the final two 30 min washes in 0.2× SSC, 0.5% SDS was increased to 60° C. Another preferred set of highly stringent conditions uses two final washes in 0.1× SSC, 0.1% SDS at 65° C.

Substantially similar nucleic acid fragments of the instant invention may also be characterized by the percent identity of the amino acid sequences that they encode to the amino acid sequences disclosed herein, as determined by algorithms commonly employed b those skilled in this art. Suitable nucleic acid fragments (isolated polynucleotides of the present invention) encode polypeptides that are at least about 70% identical, preferably at least about 80% identical to the amino acid sequences reported herein. Preferred nucleic acid fragments encode amino acid sequences that are at least about 85% identical to the amino acid sequences reported herein. More preferred nucleic acid fragments encode amino acid sequences that are at least about 90% identical to the amino acid sequences reported herein. Most preferred are nucleic acid fragments that encode amino acid sequences that are at least about 95% identical to the amino acid sequences reported herein. Suitable nucleic acid fragments not only have the above identities but typically encode a polypeptide having at least 50 amino acids, preferably at least 100 amino acids, more preferably at least 150 amino acids, still more preferably at least 200 amino acids, and most preferably at least 250 or at least 300 amino acids. Sequence alignments and percent identity calculations were performed using the Megalign program of the LASERGENE bioinformatics computing suit (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

A “substantial portion” of an amino acid or nucleotide sequence comprises an amino acid or a nucleotide sequence that is sufficient to afford putative identification of the protein or gene that the amino acid or nucleotide sequence comprises. Amino acid and nucleotide sequences can be evaluated either manually by one skilled in the art, or by using computer-based sequence comparison and identification tools that employ algorithms such as BLAST (Basic Local Alignment Search Tool; Altschul et al. (1993) J. Mol. Biol. 215:403-410; see also www.ncbi.nlm.nih.gov/BLAST/). In general, a sequence of ten or more contiguous amino acids or thirty or more contiguous nucleotides is necessary in order to putatively identify a polypeptide or nucleic acid sequence as homologous to a known protein or gene. Moreover, with respect to nucleotide sequences, gene-specific oligonucleotide probes comprising 30 or more contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12 or more nucleotides may be used as amplification primers in PCR in order to obtain a particular nucleic acid fragment comprising the primers. Accordingly, a “substantial portion” of a nucleotide sequence comprises a nucleotide sequence that will afford specific identification and/or isolation of a nucleic acid fragment comprising the sequence. The instant specification teaches amino acid and nucleotide sequences encoding polypeptides that comprise one or more particular plant proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

“Codon degeneracy” refers to divergence in the genetic code permitting variation of the nucleotide sequence without effecting the amino acid sequence of an encoded polypeptide. Accordingly, the instant invention relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for improved expression in a host cell, it is desirable to design the nucleic acid fragment such that its frequency of codon usage approaches the frequency of preferred codon usage of the host cell.

“Synthetic nucleic acid fragments” can be assembled from oligonucleotide building blocks that are chemically synthesized using procedures known to those skilled in the art. These building blocks are ligated and annealed to form larger nucleic acid fragments which may then be enzymatically assembled to construct the entire desired nucleic acid fragment. “Chemically synthesized”, as related to a nucleic acid fragment, means that the component nucleotides were assembled in vitro. Manual chemical synthesis of nucleic acid fragments may be accomplished using well established procedures, or automated chemical synthesis can be performed using one of a number of commercially available machines. Accordingly, the nucleic acid fragments can be tailored for optimal gene expression based on optimization of the nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign-gene” refers to a gene not normally found in the host organism, but that is introduced into the host organism by gene transfer. Foreign genes can comprise native genes inserted into a non-native organism, or chimeric genes. A “transgene” is a ge that has been introduced into the genome by a transformation procedure.

“Coding sequence” refers to a nucleotide sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” refers to a nucleotide sequence capable of controlling the expression of coding sequence or functional RNA. In general, a coding sequence is located 3′ to a promoter sequence. The promoter sequence consists of proximal and more distal upstream elements, the latter elements often referred to as enhancers. Accordingly, an “enhancer” is nucleotide sequence which can stimulate promoter activity and may be an innate element o the promoter or a heterologous element inserted to enhance the level or tissue-specificity o a promoter. Promoters may be derived in their entirety from a native gene, or may be composed of different elements derived from different promoters found in nature, or may even comprise synthetic nucleotide segments. It is understood by those skilled in the art th different promoters may direct the expression of a gene in different tissues or cell types, or at different stages of development, or in response to different environmental conditions. Promoters which cause a nucleic acid fragment to be expressed in most cell types at most times are commonly referred to as “constitutive promoters”. New promoters of various types useful in plant cells are constantly being discovered; numerous examples may be found in the compilation by Okamuro and Goldberg (1989) Biochemistry of Plants 15:1-82 It is further recognized that since in most cases the exact boundaries of regulatory sequence have not been completely defined, nucleic acid fragments of different lengths may have identical promoter activity.

“Translation leader sequence” refers to a nucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner and Foster (1995) Mol. Biotechnol. 3:225-236).

“3′ non-coding sequences” refer to nucleotide sequences located downstream of a coding sequence and include polyadenylation recognition sequences and other sequences encoding regulatory signals capable of affecting mRNA processing or gene expression. Th polyadenylation signal is usually characterized by affecting the addition of polyadenylic ac tracts to the 3′ end of the mRNA precursor. The use of different 3′ non-coding sequences is exemplified by Ingelbrecht et al. (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA (mRNA)” refers to the RNA that is without introns and that can be translated into polypeptides by the cell. “cDNA” refers to DNA that is complementary to and derived from an mRNA template. The cDNA can be single-stranded or converted to double stranded form using, for example, the Klenow fragment of DNA polymerase I. “Sense-RNA” refers to an RNA transcript that includes th mRNA and so can be translated into a polypeptide by the cell. “Antisense RNA” refers to an RNA transcript that is complementary to all or part of a target primary transcript or mRNA and that blocks the expression of a target gene (see U.S. Pat. No. 5,107,065, incorporated herein by reference). The complementarity of an antisense RNA may be with any part of the specific nucleotide sequence, i.e., at the 5′ non-coding sequence, 3′ non-coding sequence, introns, or the coding sequence. “Functional RNA” refers to sense RNA, antisense RNA, ribozyme RNA, or other RNA that may not be translated but yet has an effect on cellular processes.

The term “operably linked” refers to the association of two or more nucleic acid fragments on a single polynucleotide so that the function of one is affected by the other. F example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment c the invention. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression of identical or substantially similar foreig or endogenous genes (U.S. Pat. No. 5,231,020, incorporated herein by reference).

A “protein” or “polypeptide” is a chain of amino acids arranged in a specific order determined by the coding sequence in a polynucleotide encoding the polypeptide. Each protein or polypeptide has a unique function.

“Altered levels” or “altered expression” refers to the production of gene product(s). transgenic organisms in amounts or proportions that differ from that of normal or non-transformed organisms.

“Null mutant” refers here to a host cell which either lacks the expression of a certain polypeptide or expresses a polypeptide which is inactive or does not have any detectable expected enzymatic function.

“Mature protein” or the term “mature” when used in describing a protein refers to a post-translationally processed polypeptide; i.e., one from which any pre- or propeptides present in the primary translation product have been removed. “Precursor protein” or the term “precursor” when used in describing a protein refers to the primary product of translation of mRNA; i.e., with pre- and propeptides still present. Pre- and propeptides ma be but are not limited to intracellular localization signals.

A “chloroplast transit peptide” is an amino acid sequence which is translated in conjunction with a protein and directs the protein to the chloroplast or other plastid types present in the cell in which the protein is made. “Chloroplast transit sequence” refers to a nucleotide sequence that encodes a chloroplast transit peptide. A “signal peptide” is an amino acid sequence which is translated in conjunction with a protein and directs the protei to the secretory system (Chrispeels (1991) Ann. Rev. Plant Phys. Plant Mol. Biol. 42:21-53 If the protein is to be directed to a vacuole, a vacuolar targeting signal (supra) can further b added, or if to the endoplasmic reticulum, an endoplasmic reticulum retention signal (supra may be added. If the protein is to be directed to the nucleus, any signal peptide present should be removed and instead a nuclear localization signal included (Raikhel (1992) Plan Phys. 100:1627-1632).

“Transformation” refers to the transfer of a nucleic acid fragment into the genome o a host organism, resulting in genetically stable inheritance. Host organisms containing the transformed nucleic acid fragments are referred to as “transgenic” organisms. Examples of methods of plant transformation include Agrobacterium-mediated transformation (De Blae: et al. (1987) Meth. Enzymol. 143:277) and particle-accelerated or “gene gun” transformatio technology (Klein et al. (1987) Nature (London) 327:70-73; U.S. Pat. No. 4,945,050, incorporated herein by reference). Thus, isolated polynucleotides of the present invention can be incorporated into recombinant constructs, typically DNA constructs, capable of introduction into and replication in a host cell. Such a construct can be a vector that includ a replication system and sequences that are capable of transcription and translation of a polypeptide-encoding sequence in a given host cell. A number of vectors suitable for stabl transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Flevin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

“SPF1-related transcription factor” refers to a transcription factor that has at least 30%, 40%, 50%, 60%, 70%, 80%, 85%, 90%, or 95% identity with SPF1 transcription factor based on the Clustal alignment method using default parameters. Default parameters for multiple alignment of the sequences are GAP PENALTY=10, GAP LENGTH PENALTY=10. Default parameters for pairwise alignments using the Clustal method are KTUPLE 1, GAP PENALTY=3, WINDOW=5 and DIAGONALS SAVED=5.

Standard recombinant DNA and molecular cloning techniques used herein are well known in the art and are described more fully in Sambrook et al. Molecular Cloning: A Laboratory Manual; Cold Spring Harbor Laboratory Press: Cold Spring Harbor, 1989 (hereinafter “Maniatis”).

“PCR” or “polymerase chain reaction” is well known by those skilled in the art as a technique used for the amplification of specific DNA segments (U.S. Pat. Nos. 4,683,195 and 4,800,159).

The present invention concerns an isolated polynucleotide comprising: (a) a first nucleotide sequence encoding a first polypeptide comprising at least 100 amino acids, wherein the amino acid sequence of the first polypeptide and the amino acid sequence of SEQ ID NO:2 or SEQ ID NO:4 have at least 70%, 80%, 85%, 90%, or 95% identity based on the Clustal alignment method, (b) a second nucleotide sequence encoding a second polypeptide comprising at least 150 amino acids, wherein the amino acid sequence of the second polypeptide and the amino acid sequence of SEQ ID NO:12 have at least 70%, 80%, 85%, 90%, or 95% identity based on the Clustal alignment method, (c) a third nucleotide sequence encoding a third polypeptide comprising at least 250 amino acids, wherein the amino acid sequence of the third polypeptide and the amino acid sequence of SEQ ID NO:6 or SEQ ID NO:8 have at least 70%, 80%, 85%, 90%, or 95% identity based on the Clustal alignment method, (d) a fourth nucleotide sequence encoding a fourth polypeptide comprising at least 300 amino acids, wherein the amino acid sequence of the fourth polypeptide and the amino acid sequence of SEQ ID NO:10 have at least 80%, 85%, 90%, or 95% identity based on the Clustal alignment method, or (e) the complement of the first, second, third, or fourth nucleotide sequence, wherein the complement and the first, second, third, or fourth nucleotide sequence contain the same number of nucleotides and are 100% complementary. The first polypeptide preferably comprises the amino acid sequence of SEQ ID NO:2 or SEQ ID NO:4, the second polypeptide preferably comprises the amino acid sequence of SEQ ID NO:12, the third polypeptide preferably comprises the amino acid sequence of SEQ ID NO:6 or SEQ ID NO:8, and the fourth polypeptide preferably comprises the amino acid sequence of SEQ ID NO:10. The first nucleotide sequence preferably comprises the nucleotide sequence of SEQ ID NO:1 or SEQ ID NO:3, the second nucleotide sequence preferably comprises the nucleotide sequence of SEQ ID NO:11, the third nucleotide sequence preferably comprises the nucleotide sequence of SEQ ID NO:5 or SEQ ID NO:7, and the fourth nucleotide sequence preferably comprises the nucleotide sequence of SEQ ID NO:9. The first, second, third, and fourth polypeptides preferably are SPF1-related transcription factors.

Nucleic acid fragments encoding at least a portion of several SPF1-related transcription factors have been isolated and identified by comparison of random plant cDNA sequences to public databases containing nucleotide and protein sequences using the BLAST algorithms well known to those skilled in the art. The nucleic acid fragments of the instant invention may be used to isolate cDNAs and genes encoding homologous proteins from the same or other plant species. Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

For example, genes encoding other SPF1-related transcription factors, either as cDNAs or genomic DNAs, could be isolated directly by using all or a portion of the instant nucleic acid fragments as DNA hybridization probes to screen libraries from any desired plant employing methodology well known to those skilled in the art. Specific oligonucleotide probes based upon the instant nucleic acid sequences can be designed and synthesized by methods known in the art (Maniatis). Moreover, an entire sequence can be used directly to synthesize DNA probes by methods known to the skilled artisan such as random primer DNA labeling, nick translation, end-labeling techniques, or RNA probes using available in vitro transcription systems. In addition, specific primers can be designed and used to amplify a part or all of the instant sequences. The resulting amplification products can be labeled directly during amplification reactions or labeled after amplification reactions, and used as probes to isolate full length cDNA or genomic fragments under conditions of appropriate stringency.

In addition, two short segments of the instant nucleic acid fragments may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding plant genes. Alternatively, the second primer sequence may be based upon sequences derived from the cloning vector. For example, the skilled artisan can follow the RACE protocol (Frohman et al. (1988) Proc. Natl. Acad. Sci. USA 85:8998-9002) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al. (1989) Proc. Natl. Acad. Sci. USA 86:5673-5677; Loh et al. (1989) Science 243:217-220). Products generated by the 3′ and 5′ RACE procedures can be combined to generate full-length cDNAs (Frohman and Martin (1989) Techniques 1:165). Consequently, a polynucleotide comprising a nucleotide sequence of at least one of 60 (preferably one of at least 40, most preferably one of at least 30) contiguous nucleotides derived from a nucleotide sequence selected from the group consisting of SEQ ID NOs:1, 3, 5, 7, 9, and 11, and the complement of such nucleotide sequences may be used in such methods to obtain a nucleic acid fragment encoding a substantial portion of an amino acid sequence of a polypeptide.

The present invention relates to a method of obtaining a nucleic acid fragment encoding a substantial portion of an SPF1-related transcription factor polypeptide, preferably a substantial portion of a plant SPF1-related transcription factor polypeptide, comprising the steps of: synthesizing an oligonucleotide primer comprising a nucleotide sequence of at least one of 60 (preferably at least one of 40, most preferably at least one of 30) contiguous nucleotides derived from a nucleotide sequence selected from the group consisting of SEQ ID NOs:1, 3, 5, 7, 9, and 11, and the complement of such nucleotide sequences; and amplifying a nucleic acid fragment (preferably a cDNA inserted in a cloning vector) using the oligonucleotide primer. The amplified nucleic acid fragment preferably will encode a portion of an SPF1-related transcription factor polypeptide.

Availability of the instant nucleotide and deduced amino acid sequences facilitates immunological screening of cDNA expression libraries. Synthetic peptides representing portions of the instant amino acid sequences may be synthesized. These peptides can be used to immunize animals to produce polyclonal or monoclonal antibodies with specificity for peptides or proteins comprising the amino acid sequences. These antibodies can be then be used to screen cDNA expression libraries to isolate full-length cDNA clones of interest (Lerner (1984) Adv. Immunol. 36:1-34; Maniatis).

In another embodiment, this invention concerns viruses and host cells comprising either the chimeric genes of the invention as described herein or an isolated polynucleotide of the invention as described herein. Examples of host cells which can be used to practice the invention include, but are not limited to, yeast, bacteria, and plants.

As was noted above, the nucleic acid fragments of the instant invention may be used to create transgenic plants in which the disclosed polypeptides are present at higher or lower levels than normal or in cell types or developmental stages in which they are not normally found. This would have the effect of altering in those plants the level of expression of genes regulated by SPF1-related transcription factors disclosed herein which may potentially lead to changes in levels of disease resistance or seed protein accumulation.

Overexpression of the proteins of the instant invention may be accomplished by first constructing a chimeric gene in which the coding region is operably linked to a promoter capable of directing expression of a gene in the desired tissues at the desired stage of development. The chimeric gene may comprise promoter sequences and translation leader sequences derived from the same genes. 3′ Non-coding sequences encoding transcription termination signals may also be provided. The instant chimeric gene may also comprise one or more introns in order to facilitate gene expression.

Plasmid vectors comprising the instant isolated polynucleotide (or chimeric gene) may be constructed. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al. (1985) EMBO J. 4:2411-2418; De Almeida et al. (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

For some applications it may be useful to direct the instant polypeptides to different cellular compartments, or to facilitate its secretion from the cell. It is thus envisioned that the chimeric gene described above may be further supplemented by directing the coding sequence to encode the instant polypeptides with appropriate intracellular targeting sequences such as transit sequences (Keegstra (1989) Cell 56:247-253), signal sequences or sequences encoding endoplasmic reticulum localization (Chrispeels (1991) Ann. Rev. Plant Phys. Plant Mol. Biol. 42:21-53), or nuclear localization signals (Raikhel (1992) Plant Phys. 100:1627-1632) with or without removing targeting sequences that are already present. While the references cited give examples of each of these, the list is not exhaustive and more targeting signals of use may be discovered in the future.

It may also be desirable to reduce or eliminate expression of genes encoding the instant polypeptides in plants for some applications. In order to accomplish this, a chimeric gene designed for co-suppression of the instant polypeptide can be constructed by linking a gene or gene fragment encoding that polypeptide to plant promoter sequences. Alternatively, a chimeric gene designed to express antisense RNA for all or part of the instant nucleic acid fragment can be constructed by linking the gene or gene fragment in reverse orientation to plant promoter sequences. Either the co-suppression or antisense chimeric genes could be introduced into plants via transformation wherein expression of the corresponding endogenous genes are reduced or eliminated.

Molecular genetic solutions to the generation of plants with altered gene expression have a decided advantage over more traditional plant breeding approaches. Changes in plant phenotypes can be produced by specifically inhibiting expression of one or more genes by antisense inhibition or cosuppression (U.S. Pat. Nos. 5,190,931, 5,107,065 and 5,283,323). An antisense or cosuppression construct would act as a dominant negative regulator of gene activity. While conventional mutations can yield negative regulation of gene activity these effects are most likely recessive. The dominant negative regulation available with a transgenic approach may be advantageous from a breeding perspective. In addition, the ability to restrict the expression of a specific phenotype to the reproductive tissues of the plant by the use of tissue specific promoters may confer agronomic advantages relative to conventional mutations which may have an effect in all tissues in which a mutant gene is ordinarily expressed.

The person skilled in the art will know that special considerations are associated with the use of antisense or cosuppression technologies in order to reduce expression of particular genes. For example, the proper level of expression of sense or antisense genes may require the use of different chimeric genes utilizing different regulatory elements known to the skilled artisan. Once transgenic plants are obtained by one of the methods described above, it will be necessary to screen individual transgenics for those that most effectively display the desired phenotype. Accordingly, the skilled artisan will develop methods for screening large numbers of transformants. The nature of these screens will generally be chosen on practical grounds. For example, one can screen by looking for changes in gene expression by using antibodies specific for the protein encoded by the gene being suppressed, or one could establish assays that specifically measure enzyme activity. A preferred method will be one which allows large numbers of samples to be processed rapidly, since it will be expected that a large number of transformants will be negative for the desired phenotype.

In another embodiment, the present invention concerns an isolated polypeptide comprising: (a) a first amino acid sequence comprising at least 100 amino acids, wherein the first amino acid sequence and the amino acid sequence of SEQ ID NO:2 or SEQ ID NO:4 have at least 70%, 80%, 85%, 90%, or 95% identity based on the Clustal alignment method, (b) a second amino acid sequence comprising at least 150 amino acids, wherein the second amino acid sequence and the amino acid sequence of SEQ ID NO:12 have at least 70%, 80%, 85%, 90%, or 95% identity based on the Clustal alignment method, (c) a third amino acid sequence comprising at least 250 amino acids, wherein the third amino acid sequence and the amino acid sequence of SEQ ID NO:6 or SEQ ID NO:8 have at least 70%, 80%, 85%, 90%, or 95% identity based on the Clustal alignment method, or (d) a fourth amino acid sequence comprising at least 300 amino acids, wherein the fourth amino acid sequence and the amino acid sequence of SEQ ID NO:10 have at least 80%, 85%, 90%, or 95% identity based on the Clustal alignment method. The first amino acid sequence preferably comprises the amino acid sequence of SEQ ID NO:2 or SEQ ID NO:4, the second amino acid sequence preferably comprises the amino acid sequence of SEQ ID NO:12, the third amino acid sequence preferably comprises the amino acid sequence of SEQ ID NO:6 or SEQ ID NO:8, and the fourth amino acid sequence preferably comprises the amino acid sequence of SEQ ID NO:10. The polypeptide preferably is an SPF1-related transcription factor.

The instant polypeptides (or portions thereof) may be produced in heterologous host cells, particularly in the cells of microbial hosts, and can be used to prepare antibodies to these proteins by methods well known to those skilled in the art. The antibodies are useful for detecting the polypeptides of the instant invention in situ in cells or in vitro in cell extracts. Preferred heterologous host cells for production of the instant polypeptides are microbial hosts. Microbial expression systems and expression vectors containing regulatory sequences that direct high level expression of foreign proteins are well known to those skilled in the art. Any of these could be used to construct a chimeric gene for production of the instant polypeptides. This chimeric gene could then be introduced into appropriate microorganisms via transformation to provide high level expression of the encoded SPF1-related transcription factor. An example of a vector for high level expression of the instant polypeptides in a bacterial host is provided (Example 6).

All or a substantial portion of the polynucleotides of the instant invention may also be used as probes for genetically and physically mapping the genes that they are a part of, and used as markers for traits linked to those genes. Such information may be useful in plant breeding in order to develop lines with desired phenotypes. For example, the instant nucleic acid fragments may be used as restriction fragment length polymorphism (RFLP) markers. Southern blots (Maniatis) of restriction-digested plant genomic DNA may be probed with the nucleic acid fragments of the instant invention. The resulting banding patterns may then be subjected to genetic analyses using computer programs such as MapMaker (Lander et al. (1987) Genomics 1:174-181) in order to construct a genetic map. In addition, the nucleic acid fragments of the instant invention may be used to probe Southern blots containing restriction endonuclease-treated genomic DNAs of a set of individuals representing parent and progeny of a defined genetic cross. Segregation of the DNA polymorphisms is noted and used to calculate the position of the instant nucleic acid sequence in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4:37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

Nucleic acid probes derived from the instant nucleic acid sequences may also be used for physical mapping (i.e., placement of sequences on physical maps; see Hoheisel et al. In: Nonmammalian Genomic Analysis: A Practical Guide, Academic press 1996, pp. 319-346, and references cited therein).

In another embodiment, nucleic acid probes derived from the instant nucleic acid sequences may be used in direct fluorescence in situ hybridization (FISH) mapping (Trask (1991) Trends Genet. 7:149-154). Although current methods of FISH mapping favor use of large clones (several to several hundred KB; see Laan et al. (1995) Genome Res. 5:13-20), improvements in sensitivity may allow performance of FISH mapping using shorter probes.

A variety of nucleic acid amplification-based methods of genetic and physical mapping may be carried out using the instant nucleic acid sequences. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid fragment is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

Loss of function mutant phenotypes may be identified for the instant cDNA clones either by targeted gene disruption protocols or by identifying specific mutants for these genes contained in a maize population carrying mutations in all possible genes (Ballinger and Benzer (1989) Proc. Natl. Acad. Sci USA 86:9402-9406; Koes et al. (1995) Proc. Natl. Acad. Sci USA 92:8149-8153; Bensen et al. (1995) Plant Cell 7:75-84). The latter approach may be accomplished in two ways. First, short segments of the instant nucleic acid fragments may be used in polymerase chain reaction protocols in conjunction with a mutation tag sequence primer on DNAs prepared from a population of plants in which Mutator transposons or some other mutation-causing DNA element has been introduced (see Bensen, supra). The amplification of a specific DNA fragment with these primers indicates the insertion of the mutation tag element in or near the plant gene encoding the instant polypeptide. Alternatively, the instant nucleic acid fragment may be used as a hybridization probe against PCR amplification products generated from the mutation population using the mutation tag sequence primer in conjunction with an arbitrary genomic site primer, such as that for a restriction enzyme site-anchored synthetic adaptor. With either method, a plant containing a mutation in the endogenous gene encoding the instant polypeptide can be identified and obtained. This mutant plant can then be used to determine or confirm the natural function of the instant polypeptides disclosed herein.

### EXAMPLES

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The disclosure of each reference set forth herein is incorporated herein by reference in its entirety.

### Example 1

### Composition of cDNA Libraries; Isolation and Sequencing of cDNA Clones

cDNA libraries representing mRNAs from various corn (Zea mays), rice (Oryza sativa), soybean (Glycine max), and wheat (Triticum aestivum) tissues were prepared. The characteristics of the libraries are described below.

TABLE 2
cDNA Libraries from Corn, Rice, Soybean, and Wheat

| Library | Tissue | Clone |
|---|---|---|
| ci11c | Corn (EB90) Pooled Immature Leaf | ci11c.pk001.e13 |
| | Tissue at Stages V4, V6 and V8* | |
| p0128 | Corn Primary and Secondary Immature | p0128.cpiar39r |
| | Ear | |
| rlr24 | Resistant Rice Leaf 15 Days After | rlr24.pk0007.a8 |
| | Germination, 24 Hours | |
| | After Infection of Strain Magnaporthe | rlr24.pk0069.h10 |
| | grisea 4360-R-62 (AVR2-YAMO) | |
| sls1c | Soybean (variety S1990) Infected With | sls1c.pk033.c17 |
| | Sclerotinia sclerotiorum Mycelium | |
| wlmk1 | Wheat Seedling 1 Hour After Inoculation | wlmk1.pk0035.d9 |
| | With Erysiphe graminis f. sp tritici and | |
| | Treatment With Herbicide** | |

*Corn developmental stages are explained in the publication “How a corn plant develops” from the Iowa State University Coop. Ext. Service Special Report No. 48 reprinted June 1993.
**Application of 6-iodo-2-propoxy-3-propyl-4(3H)-quinazolinone; synthesis and methods of using this compound are described in USSN 08/545,827, incorporated herein by reference.

cDNA libraries may be prepared by any one of many methods available. For example, the cDNAs may be introduced into plasmid vectors by first preparing the cDNA libraries in Uni-ZAP™ XR vectors according to the manufacturer's protocol (Stratagene Cloning Systems, La Jolla, Calif.). The Uni-ZAP™ XR libraries are converted into plasmid libraries according to the protocol provided by Stratagene. Upon conversion, cDNA inserts will be contained in the plasmid vector pBluescript. In addition, the cDNAs may be introduced directly into precut Bluescript II SK(+) vectors (Stratagene) using T4 DNA ligase (New England Biolabs), followed by transfection into DH10B cells according to the manufacturer's protocol (GIBCO BRL Products). Once the cDNA inserts are in plasmid vectors, plasmid DNAs are prepared from randomly picked bacterial colonies containing recombinant pBluescript plasmids, or the insert cDNA sequences are amplified via polymerase chain reaction using primers specific for vector sequences flanking the inserted cDNA sequences. Amplified insert DNAs or plasmid DNAs are sequenced in dye-primer sequencing reactions to generate partial cDNA sequences (expressed sequence tags or “ESTs”; see Adams et al., (1991) Science 252:1651-1656). The resulting ESTs are analyzed using a Perkin Elmer Model 377 fluorescent sequencer.

Full-insert sequence (FIS) data is generated utilizing a modified transposition protocol. Clones identified for FIS are recovered from archived glycerol stocks as single colonies, and plasmid DNAs are isolated via alkaline lysis. Isolated DNA templates are reacted with vector primed M13 forward and reverse oligonucleotides in a PCR-based sequencing reaction and loaded onto automated sequencers. Confirmation of clone identification is performed by sequence alignment to the original EST sequence from which the FIS request is made.

Confirmed templates are transposed via the Primer Island transposition kit (PE Applied Biosystems, Foster City, Calif.) which is based upon the Saccharomyces cerevisiae Ty1 transposable element (Devine and Boeke (1994) Nucleic Acids Res. 22:3765-3772). The in vitro transposition system places unique binding sites randomly throughout a population of large DNA molecules. The transposed DNA is then used to transform DH10B electro-competent cells (Gibco BRL/Life Technologies, Rockville, Md.) via electroporation. The transposable element contains an additional selectable marker (named DHFR; Fling and Richards (1983) Nucleic Acids Res. 11:5147-5158), allowing for dual selection on agar plates of only those subclones containing the integrated transposon. Multiple subclones are randomly selected from each transposition reaction, plasmid DNAs are prepared via alkaline lysis, and templates are sequenced (ABI Prism dye-terminator ReadyReaction mix) outward from the transposition event site, utilizing unique primers specific to the binding sites within the transposon.

Sequence data is collected (ABI Prism Collections) and assembled using Phred/Phrap (P. Green, University of Washington, Seattle). Phrep/Phrap is a public domain software program which re-reads the ABI sequence data, re-calls the bases, assigns quality values, and writes the base calls and quality values into editable output files. The Phrap sequence assembly program uses these quality values to increase the accuracy of the assembled sequence contigs. Assemblies are viewed by the Consed sequence editor (D. Gordon, University of Washington, Seattle).

### Example 2

### Identification of cDNA Clones

cDNA clones encoding SPF1-related transcription factors were identified by conducting BLAST (Basic Local Alignment Search Tool; Altschul et al. (1993) J. Mol. Biol. 215:403-410; see also www.ncbi.nlm.nih.gov/BLAST/) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences obtained in Example 1 were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish and States (1993) Nat. Genet. 3:266-272) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

ESTs submitted for analysis are compared to the genbank database as described above. ESTs that contain sequences more 5- or 3-prime can be found by using the BLASTn algorithm (Altschul et al (1997) Nucleic Acids Res. 25:3389-3402.) against the DuPont proprietary database comparing nucleotide sequences that share common or overlapping regions of sequence homology. Where common or overlapping sequences exist between two or more nucleic acid fragments, the sequences can be assembled into a single contiguous nucleotide sequence, thus extending the original fragment in either the 5 or 3 prime direction. Once the most 5-prime EST is identified, its complete sequence can be determined by Full Insert Sequencing as described in Example 1. Homologous genes belonging to different species can be found by comparing the amino acid sequence of a known gene (from either a proprietary source or a public database) against an EST database using the tBLASTn algorithm. The tBLASTn algorithm searches an amino acid query against a nucleotide database that is translated in all 6 reading frames. This search allows for differences in nucleotide codon usage between different species, and for codon degeneracy.

### Example 3

### Characterization of cDNA Clones Encoding SPF1-Related Transcription Factors

The BLASTX search using the EST sequences from clones listed in Table 3 revealed similarity of the polypeptides encoded by the cDNAs to SPF1 protein from Ipomoea batatas (NCBI GenBank Identifier (GI) No. 1076685). Shown in Table 3 are the BLAST results for individual ESTs (“EST”), the sequences of the entire cDNA inserts comprising the indicated cDNA clones (“FIS”), the sequences of contigs assembled from two or more ESTs (“Contig”), sequences of contigs assembled from an FIS and one or more ESTs (“Contig*”), or sequences encoding an entire protein derived from an FIS, a contig, or an FIS and PCR (“CGS”):

TABLE 3
BLAST Results for Sequences Encoding Polypeptides Homologous
to SPF1 Protein

| | | BLAST pLog Score |
|---|---|---|
| | | NCBI GenBank Identifier (GI) |
| Clone | Status | No. 1076685 |
| ci11c.pk001.e13 | EST | 30.22 |
| p0128.cpiar39r | EST | 26.22 |
| rlr24.pk0007.a8 (FIS) | CGS | 107.00 |
| rlr24.pk0069.h10 (FIS) | CGS | 130.00 |
| sls1c.pk033.c17 (FIS) | CGS | >254.00 |
| wlmk1.pk0035.d9 | FIS | 90.22 |

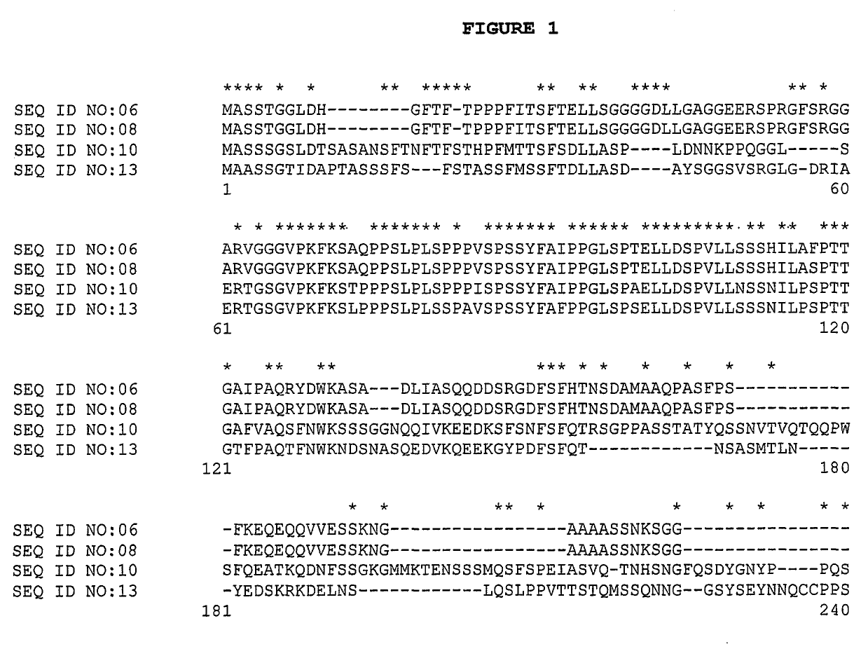

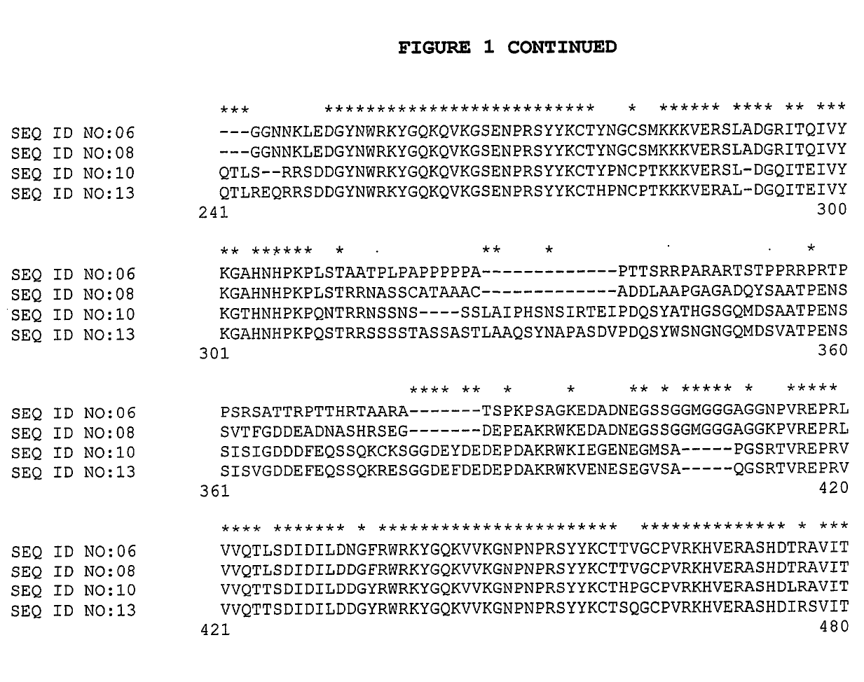

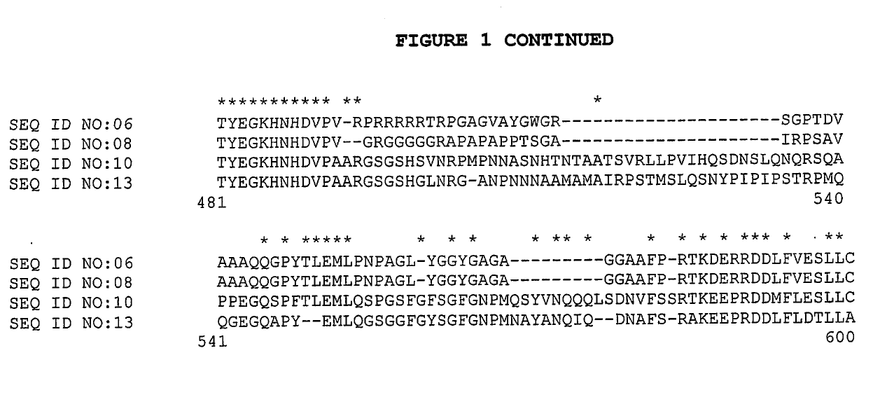

FIG. 1 presents an alignment of the amino acid sequences set forth in SEQ ID NOs:6, 8, and 10 and the Ipomoea batatas sequence (NCBI GI No. 1076685) (SEQ ID NO:13). The data in Table 4 represents a calculation of the percent identity of the amino acid sequences set forth in SEQ ID NOs:6, 8, and 10 and the Ipomoea batatas sequence (NCBI GI No. 1076685) (SEQ ID NO:13).

TABLE 4
Percent Identity of Amino Acid Sequences Deduced From the
Nucleotide Sequences of cDNA Clones Encoding Polypeptides
Homologous to SPF1 Protein

| | Percent Identity to |
|---|---|
| SEQ ID NO. | NCBI GI No. 1076685; SEQ ID NO: 13 |
| 6 | 40.8 |
| 8 | 46.8 |
| 10 | 59.9 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments and BLAST scores and probabilities indicate that the nucleic acid fragments comprising the instant cDNA clones encode a substantial portion of an SPF1-related transcription factor. These sequences represent the first corn, rice, soybean, and wheat sequences indicated to encode SPF1-related transcription factors known to Applicant. Nucleic acid fragments known to encode SPF1-related transcription factors have been previously obtained from a number of species including oat (NCBI GI No. 4894965), Pimpinella brachycarpa (NCBI GI No. 3420906), and parsley (NCBI GI No. 1431872).

### Example 4

### Expression of Chimeric Genes in Monocot Cells

A chimeric gene comprising a cDNA encoding the instant polypeptide in sense orientation with respect to the maize 27 kD zein promoter that is located 5′ to the cDNA fragment, and the 10 kD zein 3′ end that is located 3′ to the cDNA fragment, can be constructed. The cDNA fragment of this gene may be generated by polymerase chain reaction (PCR) of the cDNA clone using appropriate oligonucleotide primers. Cloning sites (NcoI or SmaI) can be incorporated into the oligonucleotides to provide proper orientation of the DNA fragment when inserted into the digested vector pML103 as described below. Amplification is then performed in a standard PCR. The amplified DNA is then digested with restriction enzymes NcoI and SmaI and fractionated on an agarose gel. The appropriate band can be isolated from the gel and combined with a 4.9 kb NcoI-SmaI fragment of the plasmid pML103. Plasmid pML103 has been deposited under the terms of the Budapest Treaty at ATCC (American Type Culture Collection, 10801 University Blvd., Manassas, Va. 20110-2209), and bears accession number ATCC 97366. The DNA segment from pML103 contains a 1.05 kb SalI-NcoI promoter fragment of the maize 27 kD zein gene and a 0.96 kb SmaI-SalI fragment from the 3′ end of the maize 10 kD zein gene in the vector pGem9Zf(+) (Promega). Vector and insert DNA can be ligated at 15° C. overnight, essentially as described (Maniatis). The ligated DNA may then be used to transform E. coli XL1-Blue (Epicurian Coli XL-1 Blue™; Stratagene). Bacterial transformants can be screened by restriction enzyme digestion of plasmid DNA and limited nucleotide sequence analysis using the dideoxy chain termination method (Sequenase™ DNA Sequencing Kit; U.S. Biochemical). The resulting plasmid construct would comprise a chimeric gene encoding, in the 5′ to 3′ direction, the maize 27 kD zein promoter, a cDNA fragment encoding the instant polypeptide, and the 10 kD zein 3′ region.

The chimeric gene described above can then be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al. (1975) Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (obtained from Dr. Peter Eckes, Hoechst Ag, Frankfurt, Germany) may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell et al. (1985) Nature 313:810-812) and the 3′ region of the nopaline synthase gene from the T-DNA of the Ti plasmid of Agrobacterium tumefaciens.

The particle bombardment method (Klein et al. (1987) Nature 327:70-73) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton™ flying disc (Bio-Rad Labs). The particles are then accelerated into the corn tissue with a Biolistic™ PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains gluphosinate (2 mg per liter) and lacks casein or proline. The tissue continues to grow slowly on this medium. After an additional 2 weeks the tissue can be transferred to fresh N6 medium containing gluphosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al. (1990) Bio/Technology 8:833-839).

### Example 5

### Expression of Chimeric Genes in Dicot Cells

A seed-specific expression cassette composed of the promoter and transcription terminator from the gene encoding the β subunit of the seed storage protein phaseolin from the bean Phaseolus vulgaris (Doyle et al. (1986) J. Biol. Chem. 261:9228-9238) can be used for expression of the instant polypeptides in transformed soybean. The phaseolin cassette includes about 500 nucleotides upstream (5′) from the translation initiation codon and about 1650 nucleotides downstream (3′) from the translation stop codon of phaseolin. Between the 5′ and 3′ regions are the unique restriction endonuclease sites Nco I (which includes the ATG translation initiation codon), Sma I, Kpn I and Xba I. The entire cassette is flanked by Hind III sites.

The cDNA fragment of this gene may be generated by polymerase chain reaction (PCR) of the cDNA clone using appropriate oligonucleotide primers. Cloning sites can be incorporated into the oligonucleotides to provide proper orientation of the DNA fragment when inserted into the expression vector. Amplification is then performed as described above, and the isolated fragment is inserted into a pUC18 vector carrying the seed expression cassette.

Soybean embryos may then be transformed with the expression vector comprising sequences encoding the instant polypeptides. To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos which produce secondary embryos are then excised and placed into a suitable liquid medium. After repeated selection for clusters of somatic embryos which multiplied as early, globular staged embryos, the suspensions are maintained as described below.

Soybean embryogenic suspension cultures can be maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium.

Soybean embryogenic suspension cultures may then be transformed by the method of particle gun bombardment (Klein et al. (1987) Nature (London) 327:70-73, U.S. Pat. No. 4,945,050). A DuPont Biolistic™ PDS1000/HE instrument (helium retrofit) can be used for these transformations.

A selectable marker gene which can be used to facilitate soybean transformation is a chimeric gene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188) and the 3′ region of the nopaline synthase gene from the T-DNA of the Ti plasmid of Agrobacterium tumefaciens. The seed expression cassette comprising the phaseolin 5′ region, the fragment encoding the instant polypeptide and the phaseolin 3′ region can be isolated as a restriction fragment. This fragment can then be inserted into a unique restriction site of the vector carrying the marker gene.

To 50 μL of a 60 mg/mL 1 μm gold particle suspension is added (in order): 5 μL DNA (1 μg/μL), 20 μL spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 6

### Expression of Chimeric Genes in Microbial Cells

The cDNAs encoding the instant polypeptides can be inserted into the T7 E. coli expression vector pBT430. This vector is a derivative of pET-3a (Rosenberg et al. (1987) Gene 56:125-135) which employs the bacteriophage T7 RNA polymerase/T7 promoter system. Plasmid pBT430 was constructed by first destroying the EcoR I and Hind III sites in pET-3a at their original positions. An oligonucleotide adaptor containing EcoR I and Hind III sites was inserted at the BamH I site of pET-3a. This created pET-3aM with additional unique cloning sites for insertion of genes into the expression vector. Then, the Nde I site at the position of translation initiation was converted to an Nco I site using oligonucleotide-directed mutagenesis. The DNA sequence of pET-3aM in this region, 5′-CATATGG, was converted to 5′-CCCATGG in pBT430.

Plasmid DNA containing a cDNA may be appropriately digested to release a nucleic acid fragment encoding the protein. This fragment may then be purified on a 1% low melting agarose gel. Buffer and agarose contain 10 μg/ml ethidium bromide for visualization of the DNA fragment. The fragment can then be purified from the agarose gel by digestion with GELase™ (Epicentre Technologies, Madison, Wis.) according to the manufacturer's instructions, ethanol precipitated, dried and resuspended in 20 μL of water. Appropriate oligonucleotide adapters may be ligated to the fragment using T4 DNA ligase (New England Biolabs (NEB), Beverly, Mass.). The fragment containing the ligated adapters can be purified from the excess adapters using low melting agarose as described above. The vector pBT430 is digested, dephosphorylated with alkaline phosphatase (NEB) and deproteinized with phenol/chloroform as described above. The prepared vector pBT430 and fragment can then be ligated at 16° C. for 15 hours followed by transformation into DH5 electrocompetent cells (GIBCO BRL). Transformants can be selected on agar plates containing LB media and 100 μg/mL ampicillin. Transformants containing the gene encoding the instant polypeptide are then screened for the correct orientation with respect to the T7 promoter by restriction enzyme analysis.

For high level expression, a plasmid clone with the cDNA insert in the correct orientation relative to the T7 promoter can be transformed into E. coli strain BL21(DE3) (Studier et al. (1986) J. Mol. Biol. 189:113-130). Cultures are grown in LB medium containing ampicillin (100 mg/L) at 25° C. At an optical density at 600 nm of approximately 1, IPTG (isopropylthio-β-galactoside, the inducer) can be added to a final concentration of 0.4 mM and incubation can be continued for 3 h at 25°. Cells are then harvested by centrifugation and re-suspended in 50 μL of 50 mM Tris-HCl at pH 8.0 containing 0.1 mM DTT and 0.2 mM phenyl methylsulfonyl fluoride. A small amount of 1 mm glass beads can be added and the mixture sonicated 3 times for about 5 seconds each time with a microprobe sonicator. The mixture is centrifuged and the protein concentration of the supernatant determined. One μg of protein from the soluble fraction of the culture can be separated by SDS-polyacrylamide gel electrophoresis. Gels can be observed for protein bands migrating at the expected molecular weight.

### Example 7

### Generation of Transgenic Rice Plants Containing Chimeric Gene Encoding SPF1-Related Transcription Factor

A BamHI site was introduced into clone rlr24.pk0007.a8 via site-directed mutagenesis using oligonucleotide Q107 and the U.F.E. mutagenesis kit (Amersham Pharmacia Biotech) according to the method of Amersham Pharmacia Biotech, to generate pSPF1-B.

Q107: 5′-GAAAAATTCATCAGTGGATCCTTATTTGACCTGTCT-3′ (SEQ ID NO:14)

The BamHI fragment of pSPF1-B which contains the full-length coding region of rice SPF1-related transcription factor-encoding gene, was ligated into pAHC17 (Christensen and Quail (1996) Transgenic Research 5:213-218) cut with BamHI to generate pQZ2001. Insertion of the pSPF1-B fragment in the proper orientation and correct fusion region between the corn ubiquitin promoter in pAHC17 and the pSPF1-B fragment were determined by sequence analysis.

For rice transformation, pQZ2001 (described above) and pML18 were used. The bacterial hygromycin B phosphotransferase (Hpt II) gene from Streptomyces hygroscopicus that confers resistance to the antibiotic was used as the selectable marker for rice transformation. In the vector that was used, pML18, the Hpt II gene was engineered with the 35S promoter from Cauliflower Mosaic Virus and the termination and polyadenylation signals from the octopine synthase gene of Agrobacterium tumefaciens. pML18 is described in WO 97/47731, which was published on Dec. 18, 1997, the disclosure of which is hereby incorporated by reference.

Embryogenic callus cultures derived from the scutellum of germinating Nipponbare seeds served as source material for transformation experiments. This material was generated by germinating sterile rice seeds on a callus initiation media (MS salts, Nitsch and Nitsch vitamins, 1.0 mg/l 2,4-D and 10 μM AgNO₃) in the dark at 27-28° C. Embryogenic callus proliferating from the scutellum of the embryos was the transferred to CM media (N6 salts, Nitsch and Nitsch vitamins, 1 mg/l 2,4-D, Chu et al., 1985, Sci. Sinica 18: 659-668). Callus cultures were maintained on CM by routine sub-culture at two week intervals and used for transformation within 10 weeks of initiation.

Callus was prepared for transformation by subculturing 0.5-1.0 mm pieces approximately 1 mm apart, arranged in a circular area of about 4 cm in diameter, in the center of a circle of Whatman #541 paper placed on CM media. The plates with callus were incubated in the dark at 27-28° C. for 3-5 days. Prior to bombardment, the filters with callus were transferred to CM supplemented with 0.25 M mannitol and 0.25 M sorbitol for 3 hr. in the dark. The petri dish lids were then left ajar for 20-45 minutes in a sterile hood to allow moisture on tissue to dissipate.

Circular plasmid DNA of two different plasmids, pML18 containing the selectable marker for rice transformation and pQZ2001, were co-precipitated onto the surface of gold particles. To accomplish this, a total of 10 μg of DNA at a 2:1 ratio of trait:selectable marker DNAs were added to 50 μl aliquot of gold particles that had been resuspended at a concentration of 60 mg ml⁻¹. Calcium chloride (50 μl of a 2.5 M solution) and spermidine (20 μl of a 0.1 M solution) were then added to the gold-DNA suspension as the tube was vortexing for 3 min. The gold particles were centrifuged in a microfuge for 1 sec and the supernatant removed. The gold particles were then washed twice with 1 ml of absolute ethanol and then resuspended in 50 μl of absolute ethanol and sonicated (bath sonicator) for one second to disperse the gold particles. The gold suspension was incubated at −70° C. for five minutes and sonicated (bath sonicator) if needed to disperse the particles. Six μl of the DNA-coated gold particles were then loaded onto mylar macrocarrier disks and the ethanol was allowed to evaporate.

At the end of the drying period, a petri dish containing the tissue was placed in the chamber of the PDS-1000/He. The air in the chamber was then evacuated to a vacuum of 28-29 inches Hg. The macrocarrier was accelerated with a helium shock wave using a rupture membrane that bursts when the He pressure in the shock tube reaches 1080-1100 psi. The tissue was placed approximately 8 cm from the stopping screen and the callus was bombarded two times. Five to seven plates of tissue were bombarded in this way with the DNA-coated gold particles. Following bombardment, the callus tissue was transferred to CM media without supplemental sorbitol or mannitol.

Within 3-5 days after bombardment the callus tissue was transferred to SM media (CM medium containing 50 mg/l hygromycin). To accomplish this, callus tissue was transferred from plates to sterile 50 ml conical tubes and weighed. Molten top-agar at 40° C. was added using 2.5 ml of top agar/100 mg of callus. Callus clumps were broken into fragments of less than 2 mm diameter by repeated dispensing through a 10 ml pipet. Three ml aliquots of the callus suspension were plated onto fresh SM media and the plates were incubated in the dark for 4 weeks at 27-28° C. After 4 weeks, transgenic callus events were identified, transferred to fresh SM plates and grown for an additional 2 weeks in the dark at 27-28° C.

Growing callus was transferred to RM1 media (MS salts, Nitsch and Nitsch vitamins, 2% sucrose, 3% sorbitol, 0.4% gelrite+50 ppm hyg B) for 2 weeks in the dark at 25° C. After 2 weeks the callus was transferred to RM2 media (MS salts, Nitsch and Nitsch vitamins, 3% sucrose, 0.4% gelrite+50 ppm hyg B) and placed under cool white light (˜40 μEm⁻²s⁻¹) with a 12 hr photoperiod at 25° C. and 30-40% humidity. After 2-4 weeks in the light, callus began to organize, and form shoots. Shoots were removed from surrounding callus/media and gently transferred to RM3 media (1/2× MS salts, Nitsch and Nitsch vitamins, 1% sucrose+50 ppm hygromycin B) in phytatrays (Sigma Chemical Co., St. Louis, Mo.) and incubation was continued using the same conditions as described in the previous step.

Plants were transferred from RM3 to 4″ pots containing Metro mix 350 after 2-3 weeks, when sufficient root and shoot growth had occurred. Plants were grown using a 12 hr/12 hr light/dark cycle using ˜30/18° C. day/night temperature regimen.

Thirty lines of hygromycin resistant rice were generated. PCR analysis demonstrated that 26 of them were transgenic lines. Northern blot analysis showed that there were at least 5 high level expressers of the introduced chimeric gene (resulting expression is more than 10 times the expression in untransformed rice) and at least 3 medium level expressers (resulting expression is about 5 to 10 times the expression in untransformed rice).

What is claimed is:

1. An isolated polynucleotide comprising:
(a) a first nucleotide sequence encoding a first polypeptide comprising at least 100 amino acids, wherein the amino acid sequence of the first polypeptide and the amino acid sequence of SEQ ID NO:2 or SEQ ID NO:4 have at least 70% identity based on the Clustal alignment method,
(b) a second nucleotide sequence encoding a second polypeptide comprising at least 150 amino acids, wherein the amino acid sequence of the second polypeptide and the amino acid sequence of SEQ ID NO:12 have at least 70% identity based on the Clustal alignment method,
(c) a third nucleotide sequence encoding a third polypeptide comprising at least 250 amino acids, wherein the amino acid sequence of the third polypeptide and the amino acid sequence of SEQ ID NO:6 or SEQ ID NO:8 have at least 70% identity based on the Clustal alignment method,
(d) a fourth nucleotide sequence encoding a fourth polypeptide comprising at least 300 amino acids, wherein the amino acid sequence of the fourth polypeptide and the amino acid sequence of SEQ ID NO:10 have at least 80% identity based on the Clustal alignment method, or
(e) the complement of the first, second, third, or fourth nucleotide sequence, wherein the complement and the first, second, third, or fourth nucleotide sequence contain the same number of nucleotides and are 100% complementary.

2. The polynucleotide of claim 1, wherein the amino acid sequence of the first polypeptide and the amino acid sequence of SEQ ID NO:2 or SEQ ID NO:4 have at least 80% identity based on the Clustal alignment method, wherein the amino acid sequence of the second polypeptide and the amino acid sequence of SEQ ID NO:12 have at least 80% identity based on the Clustal alignment method, and wherein the amino acid sequence of the third polypeptide and the amino acid sequence of SEQ ID NO:6 or SEQ ID NO:8 have at least 80% identity based on the Clustal alignment method.

3. The polynucleotide of claim 1, wherein the amino acid sequence of the first polypeptide and the amino acid sequence of SEQ ID NO:2 or SEQ ID NO:4 have at least 85% identity based on the Clustal alignment method, wherein the amino acid sequence of the second polypeptide and the amino acid sequence of SEQ ID NO:12 have at least 85% identity based on the Clustal alignment method, wherein the amino acid sequence of the third polypeptide and the amino acid sequence of SEQ ID NO:6 or SEQ ID NO:8 have at least 85% identity based on the Clustal alignment method, and wherein the amino acid sequence of the fourth polypeptide and the amino acid sequence of SEQ ID NO:10 have at least 85% identity based on the Clustal alignment method.

4. The polynucleotide of claim 1, wherein the amino acid sequence of the first polypeptide and the amino acid sequence of SEQ ID NO:2 or SEQ ID NO:4 have at least 90% identity based on the Clustal alignment method, wherein the amino acid sequence of the second polypeptide and the amino acid sequence of SEQ ID NO:12 have at least 90% identity based on the Clustal alignment method, wherein the amino acid sequence of the third polypeptide and the amino acid sequence of SEQ ID NO:6 or SEQ ID NO:8 have at least 90% identity based on the Clustal alignment method, and wherein the amino acid sequence of the fourth polypeptide and the amino acid sequence of SEQ ID NO:10 have at least 90% identity based on the Clustal alignment method.

5. The polynucleotide of claim 1, wherein the amino acid sequence of the first polypeptide and the amino acid sequence of SEQ ID NO:2 or SEQ ID NO:4 have at least 95% identity based on the Clustal alignment method, wherein the amino acid sequence of the second polypeptide and the amino acid sequence of SEQ ID NO:12 have at least 95% identity based on the Clustal alignment method, wherein the amino acid sequence of the third polypeptide and the amino acid sequence of SEQ ID NO:6 or SEQ ID NO:8 have at least 95% identity based on the Clustal alignment method, and wherein the amino acid sequence of the fourth polypeptide and the amino acid sequence of SEQ ID NO:10 have at least 95% identity based on the Clustal alignment method.

6. The isolated polynucleotide of claim 1, wherein the first polypeptide comprises the amino acid sequence of SEQ ID NO:2 or SEQ ID NO:4, wherein the second polypeptide comprises the amino acid sequence of SEQ ID NO:12, wherein the third polypeptide comprises the amino acid sequence of SEQ ID NO:6 or SEQ ID NO:8, and wherein the fourth polypeptide comprises the amino acid sequence of SEQ ID NO:10.

7. The isolated polynucleotide of claim 1, wherein the first nucleotide sequence comprises the nucleotide sequence of SEQ ID NO:1 or SEQ ID NO:3, wherein the second nucleotide sequence comprises the nucleotide sequence of SEQ ID NO:11, wherein the third nucleotide sequence comprises the nucleotide sequence of SEQ ID NO:5 or SEQ ID NO:7, and wherein the fourth nucleotide sequence comprises the nucleotide sequence of SEQ ID NO:9.

8. The isolated polynucleotide of claim 1, wherein the first, second, third, and fourth polypeptides are SPF1-related transcription factors.

9. A chimeric gene comprising the polynucleotide of any of claims 1-8 operably linked to a regulatory sequence.

10. A vector comprising the polynucleotide of any of claims 1-8.

11. An isolated polynucleotide fragment comprising a nucleotide sequence comprised by the polynucleotide of any of claims 1-8, wherein the nucleotide sequence contains at least 30 nucleotides.

12. The fragment of claim 11, wherein the nucleotide sequence contains at least 40 nucleotides.

13. The fragment of claim 11, wherein the nucleotide sequence contains at least 60 nucleotides.

14. An isolated polypeptide comprising:
(a) a first amino acid sequence comprising at least 100 amino acids, wherein the first amino acid sequence and the amino acid sequence of SEQ ID NO:2 or SEQ ID NO:4 have at least 70% identity based on the Clustal alignment method,
(b) a second amino acid sequence comprising at least 150 amino acids, wherein the second amino acid sequence and the amino acid sequence of SEQ ID NO:12 have at least 70% identity based on the Clustal alignment method,
(c) a third amino acid sequence comprising at least 250 amino acids, wherein the third amino acid sequence and the amino acid sequence of SEQ ID NO:6 or SEQ ID NO:8 have at least 70% identity based on the Clustal alignment method, or
(d) a fourth amino acid sequence comprising at least 300 amino acids, wherein the fourth amino acid sequence and the amino acid sequence of SEQ ID NO:10 have at least 80% identity based on the Clustal alignment method.

15. The polypeptide of claim 14, wherein the first amino acid sequence and the amino acid sequence of SEQ ID NO:2 or SEQ ID NO:4 have at least 80% identity based on the Clustal alignment method, wherein the second amino acid sequence and the amino acid sequence of SEQ ID NO:12 have at least 80% identity based on the Clustal alignment method, and wherein the third amino acid sequence and the amino acid sequence of SEQ ID NO:6 or SEQ ID NO:8 have at least 80% identity based on the Clustal alignment method.

16. The polypeptide of claim 14, wherein the first amino acid sequence and the amino acid sequence of SEQ ID NO:2 or SEQ ID NO:4 have at least 85% identity based on the Clustal alignment method, wherein the second amino acid sequence and the amino acid sequence of SEQ ID NO:12 have at least 85% identity based on the Clustal alignment method, wherein the third amino acid sequence and the amino acid sequence of SEQ ID NO:6 or SEQ ID NO:8 have at least 85% identity based on the Clustal alignment method, and wherein the fourth amino acid sequence and the amino acid sequence of SEQ ID NO:10 have at least 85% identity based on the Clustal alignment method.

17. The polypeptide of claim 14, wherein the first amino acid sequence and the amino acid sequence of SEQ ID NO:2 or SEQ ID NO:4 have at least 90% identity based on the Clustal alignment method, wherein the second amino acid sequence and the amino acid sequence of SEQ ID NO:12 have at least 90% identity based on the Clustal alignment method, wherein the third amino acid sequence and the amino acid sequence of SEQ ID NO:6 or SEQ ID NO:8 have at least 90% identity based on the Clustal alignment method, and wherein the fourth amino acid sequence and the amino acid sequence of SEQ ID NO:10 have at least 90% identity based on the Clustal alignment method.

18. The polypeptide of claim 14, wherein the first amino acid sequence and the amino acid sequence of SEQ ID NO:2 or SEQ ID NO:4 have at least 95% identity based on the Clustal alignment method, wherein the second amino acid sequence and the amino acid sequence of SEQ ID NO:12 have at least 95% identity based on the Clustal alignment method, wherein the third amino acid sequence and the amino acid sequence of SEQ ID NO:6 or SEQ ID NO:8 have at least 95% identity based on the Clustal alignment method, and wherein the fourth amino acid sequence and the amino acid sequence of SEQ ID NO:10 have at least 95% identity based on the Clustal alignment method.

19. The polypeptide of claim 14, wherein the first amino acid sequence comprises the amino acid sequence of SEQ ID NO:2 or SEQ ID NO:4, wherein the second amino acid sequence comprises the amino acid sequence of SEQ ID NO:12, wherein the third amino acid sequence comprises the amino acid sequence of SEQ ID NO:6 or SEQ ID NO:8, and wherein the fourth amino acid sequence comprises the amino acid sequence of SEQ ID NO:10.

20. The polypeptide of claim 14, wherein the polypeptide is an SPF1-related transcription factor.

21. A method for transforming a cell comprising introducing the polynucleotide of any of claims 1-8 into a cell.

22. A cell comprising the chimeric gene of claim 9.

23. A method for producing a transgenic plant comprising transforming a plant cell with the polynucleotide of any of claims 1-8 and regenerating a plant from the transformed plant cell.

24. A plant comprising the chimeric gene of claim 9.

25. A seed comprising the chimeric gene of claim 9.


SEQUENCE LISTING (14 sequences)

SEQ ID NO: 1 - DNA, 512 bp, Zea mays
  unsure (368): n=a,c,g or t
ctataattggcgcaaatatggacagaagcatgtcaagggaagtgaaaatcctagaagttattacaagtgcactcatcctaattgtgaagttaaaaagctattagagcgttcgcttgatggtcagattactgaagttgtttataaagggcatcataatcatcccaagccccaaccaaatagaaggttagctgctggtgcagttccttcaagccaggctgaagaaagatacgatggtgtggcacctattgaagacaaaccttcaaatatttattccaacctctgtaaccaagcacattcagctggcatggttgataatgttccgggtccagcaagtgatgatgatgttgatgctggaggtggaagacccnaccctggggggatgactcnaatgatgatgatgacttnggnctcaaaaacgcaaggaaaatggaatctgccgggtatncnatgccnggctttgantggggtaaaccaaatcccggagccccnnnttccttttnaaaaactgtttt

SEQ ID NO: 2 - protein, 170 aa, Zea mays
  unsure (123): Xaa = ANY AMINO ACID
YNWRKYGQKHVKGSENPRSYYKCTHPNCEVKKLLERSLDGQITEVVYKGHHNHPKPQPNRRLAAGAVPSSQAEERYDGVAPIEDKPSNIYSNLCNQAHSAGMVDNVPGPASDDDVDAGGGRPXPGGMTXMMMMTXXSKTQGKWNLPGXXCXALXGVNQIPEPXXPFXKLF

SEQ ID NO: 3 - DNA, 717 bp, Zea mays
  unsure (11): n=a,c,g or t
gacgcaccccntctnttctctccccctctcgtctcgtccagtcgtccccctctcccccactctacccagctgccctgctctgcgtcgctcgccatggcgtcctccacggggagcttggagcacggggggttcacgttcacgccgccgcccttcatcacctccttcaccgagctgctctccggcgcaggggacatgctaggagccggcgccgatcaggagcggtcgtcgccgagggggctgttccaccgcggcgccaggggcgtgcccaagttcaagtcggcgcagcctcccagcctgcccatctcgccgccgcccatgtcgccttcctcctacttcgccatcccgcccgggctcagccccgccgagctgctcgactcgcccgtcctgctccactcgtcctccaacatcctggcgtctcccaccactggcgccatcccggcgcagaggttcgactggaagaaggccgccgacctgatcgcgtctcagtctcagcaagacggcgacagccgggctgccgccgccggcttcgacgacttctccttcacacgggcaccttcaacgccgtgcgcgcgcacacgacgacgacgtccttaccttcatgaagaagaangtggagagggccctggccgacgggcgcatnaacgcaaaatcgngtacaaaggcgcggcacaaacaacccnaagccggttgtncacgcgccggnaacttaattccgga

SEQ ID NO: 4 - protein, 150 aa, Zea mays
MASSTGSLEHGGFTFTPPPFITSFTELLSGAGDMLGAGADQERSSPRGLFHRGARGVPKFKSAQPPSLPISPPPMSPSSYFAIPPGLSPAELLDSPVLLHSSSNILASPTTGAIPAQRFDWKKAADLIASQSQQDGDSRAAAAGFDDFSF

SEQ ID NO: 5 - DNA, 1961 bp, Oryza sativa
agtcgtctcgttctcgtctccgatcactctcctcctcatcttcgtcacggtctcctcgcttcgctagctcgcttgcttgctggctgagctgtggtacgctcgccatggcgtcctcgacgggggggttggaccacgggttcacgttcacgccgccgccgttcatcacgtcgttcaccgagctgctgtcggggggcggtggggacctgctcggcgccggcggtgaggagcgctcgccgagggggttctccagaggcggagcgagggtgggcggcggggtgcccaagttcaagtccgcgcagccgccgagcctgccgctctcgccgccgccggtgtcgccgtcgtcctacttcgccatcccgccggggctcagccccaccgagctgctcgactcccccgtcctcctcagctcctcccatatcttggcgttcccgaccaccggtgcaatcccggctcagaggtacgactggaaggccagcgccgatctcatcgcttctcagcaagatgacagccgcggcgacttctccttccacaccaactccgacgccatggccgcgcaaccggcctctttcccttccttcaaggagcaagagcagcaagtggtcgagtcgagcaagaacggcgccgccgccgcgtcgagcaacaagagcggcggcggcgggaacaacaagctggaggacgggtacaactggaggaagtacgggcagaagcaggtgaaggggagcgagaacccgaggagctactacaagtgcacctacaacggctgctccatgaagaagaaggtggagcgctcgctcgccgacggccgcatcacccagatcgtctacaagggcgcacacaaccaccccaagccgctctccaccgccgcaacgcctcttccggctccaccgccgccgcctgcgccgacgacctcgcggcgcccggcgcgggcgcggaccagtactccgccgcgacgcccgagaactcctccgtcacgttcggcgacgacgaggccgacaacgcatcgcaccgcagcgagggcgacgagcccgaagccaagcgctggaaaggaggatgctgacaacgagggcagctccggcggcatgggcggcggcgccggcggcaacccggtgcgcgagccgaggcttgtggtgcagacgctgagcgacatcgacatcctcgacaacggcttccggtggaggaagtacggccagaaggtcgtcaagggcaaccccaacccaaggagctactacaagtgcacgacggtgggctgcccggtgcggaagcacgtggagcgggcgtcgcacgacacgcgcgccgtgatcaccacctacgagggcaagcacaaccacgacgtcccggtacggccgcggcggcggcggcggacgcgccccggcgccggcgtcgcctacggctgggggcgatccgggccgacggacgtcgccgccgcccagcaggggccctacaccctcgagatgctccccaaccccgccggcctctacggcggctacggcgccggcgccggcggcgccgcgttcccgcgcaccaaggacgagcggcgggacgacctgttcgtcgagtcgctcctctgctagtcgagccgagccgagccgagctgagctgggccccacatccccctgctcgccacgtggcgtattttcgcctcgccgtatacgtacggccgtatagcgtacgtatacacgctcgcacgccctgcccaacacggcaatacacacatacatactctcgtacacacgtagtagcatacatatacagtatagtaggtggtagtggtagctagctagggagtgagatccaatttgttgattcgttgcaggccactgccacgtgggccacaccggaaacagtacacgcgtatacaccacacttgggatacgcgtacgtacgcacatgtacacgtagttttgtgcctttgtaactgctgagagacaggtcaaataagactgatgaatttttcatttctt

SEQ ID NO: 6 - protein, 488 aa, Oryza sativa
MASSTGGLDHGFTFTPPPFITSFTELLSGGGGDLLGAGGEERSPRGFSRGGARVGGGVPKFKSAQPPSLPLSPPPVSPSSYFAIPPGLSPTELLDSPVLLSSSHILAFPTTGAIPAQRYDWKASADLIASQQDDSRGDFSFHTNSDAMAAQPASFPSFKEQEQQVVESSKNGAAAASSNKSGGGGNNKLEDGYNWRKYGQKQVKGSENPRSYYKCTYNGCSMKKKVERSLADGRITQIVYKGAHNHPKPLSTAATPLPAPPPPPAPTTSRRPARARTSTPPRRPRTPPSRSATTRPTTHRTAARATSPKPSAGKEDADNEGSSGGMGGGAGGNPVREPRLVVQTLSDIDILDNGFRWRKYGQKVVKGNPNPRSYYKCTTVGCPVRKHVERASHDTRAVITTYEGKHNHDVPVRPRRRRRTRPGAGVAYGWGRSGPTDVAAAQQGPYTLEMLPNPAGLYGGYGAGAGGAAFPRTKDERRDDLFVESLLC

SEQ ID NO: 7 - DNA, 2086 bp, Oryza sativa
tcgtctcgttctcgtctccgatcactctcctcctcatcttcgtcacggtctcctcgcttcgctagctcgcttgcttgctggctgagctgtggtacgctcgccatggcgtcctcgacgggggggttggaccacgggttcacgttcacgccgccgccgttcatcacgtcgttcaccgagctgctgtcggggggcggtggggacctgctcggcgccggcggtgaggagcgctcgccgagggggttctccagaggcggagcgagggtgggcggcggggtgcccaagttcaagtccgcgcagccgccgagcctgccgctctcgccgccgccggtgtcgccgtcgtcctacttcgccatcccgccggggctcagccccaccgagctgctcgactcccccgtcctcctcagctcctcccatatcttggcgtccccgaccaccggtgcaatcccggctcagaggtacgactggaaggccagcgccgatctcatcgcttctcagcaagatgacagccgcggcgacttctccttccacaccaactccgacgccatggccgcgcaaccggcctctttcccttccttcaaggagcaagagcagcaagtggtcgagtcgagcaagaacggcgccgccgccgcgtcgagcaacaagagcggcggcggcgggaacaacaagctggaggacgggtacaactggaggaagtacgggcagaagcaggtgaaggggagcgagaacccgaggagctactacaagtgcacctacaacggctgctccatgaagaagaaggtggagcgctcgctcgccgacggccgcatcacccagatcgtctacaagggcgcacacaaccaccccaagccgctctccacccgccgcaacgcctcctcctgcgccaccgccgccgcctgcgccgacgacctcgcggcgcccggcgcgggcgcggaccagtactccgccgcgacgcccgagaactcctccgtcacgttcggcgacgacgaggccgacaacgcatcgcaccgcagcgagggcgacgagcccgaagccaagcgctggaaggaggatgctgacaacgagggcagctccggcggcatgggcggcggcgccggcggcaagccggtgcgcgagccgaggcttgtggtgcagacgctgagcgacatcgacatcctcgacgacggcttccggtggaggaagtacggccagaaggtcgtcaagggcaaccccaacccaaggagctactacaagtgcacgacggtgggctgcccggtgcggaagcacgtggagcgggcgtcgcacgacacgcgcgccgtgatcaccacctacgagggcaagcacaaccacgacgtcccggtcggccgcggcggcggcggcggacgcgccccggcgccggcgccgccgacgtcgggggcgatccggccgtcggccgtcgccgccgcccagcaggggccctacaccctcgagatgctccccaaccccgccggcctctacggcggctacggcgccggcgccggcggcgccgcgttcccgcgcaccaaggacgagcggcgggacgacctgttcgtcgagtcgctcctctgctagtcgagccgagccgagccgagctgagctgggccccacatccccctgctcgccacgtggcgtattttcgcctcgccgtatacgtacggccgtatagcgtacgtatacacgctcgcacgccctgcccaacacggcaatacacacatacatactctcgtacacacgtagtagcatacatatacagtatagtaggtggtagtggtagctagctagggagtgagatccaatttgttgattcgttgcaggccactgccacgtgggccacaccggaaacagtacacgcgtatacaccacacttgggatacgcgtacgtacgcacatgtacacgtagttttgtgcctttgtaactgctgagagacaggtcaaataagactgatgaatttttcatttcttaaaattccactcgtgtgaattactagtagtataaatatctatacatgatgtttttacaatctgtaccgaactgagaaagaggaaaaaaaagagagagatttttttttaaaaaaaaaaaaaaaaaaaaaaa

SEQ ID NO: 8 - protein, 487 aa, Oryza sativa
MASSTGGLDHGFTFTPPPFITSFTELLSGGGGDLLGAGGEERSPRGFSRGGARVGGGVPKFKSAQPPSLPLSPPPVSPSSYFAIPPGLSPTELLDSPVLLSSSHILASPTTGAIPAQRYDWKASADLIASQQDDSRGDFSFHTNSDAMAAQPASFPSFKEQEQQVVESSKNGAAAASSNKSGGGGNNKLEDGYNWRKYGQKQVKGSENPRSYYKCTYNGCSMKKKVERSLADGRITQIVYKGAHNHPKPLSTRRNASSCATAAACADDLAAPGAGADQYSAATPENSSVTFGDDEADNASHRSEGDEPEAKRWKEDADNEGSSGGMGGGAGGKPVREPRLVVQTLSDIDILDDGFRWRKYGQKVVKGNPNPRSYYKCTTVGCPVRKHVERASHDTRAVITTYEGKHNHDVPVGRGGGGGRAPAPAPPTSGAIRPSAVAAAQQGPYTLEMLPNPAGLYGGYGAGAGGAAFPRTKDERRDDLFVESLLC

SEQ ID NO: 9 - DNA, 1928 bp, Glycine max
gcacgagtctcatggcatcttcttctggtagtttagacacctctgcaagtgcaaactccttcaccaacttcaccttctccacacaccctttcatgaccacttctttctctgacctccttgcttctcccttggacaacaacaagccaccacagggtggtttgtctgagagaactggctctggtgttcccaaattcaagtccacaccaccaccttctctgcctctctctccccctcccatttctccttcttcttactttgctattcctcctggtttgagccctgctgagcttcttgactcgccggttctccttaactcttccaacattctgccatctccaacaactggagcatttgttgctcagagcttcaattggaagagcagttcaggggggaatcagcaaattgtcaaggaagaagacaaaagcttctcaaatttctctttccaaacccgatcaggacctcctgcttcatccacagcaacataccagtcttcaaatgtcacagttcaaacacaacagccatggagttttcaggaggccacgaaacaggataatttttcctcaggaaagggtatgatgaaaactgaaaactcttcttccatgcagagtttttcccctgagattgctagtgtccaaactaaccatagcaatgggtttcaatccgattatggcaattaccccccacaatctcagactttaagtagaaggtcagatgatgggtacaattggaggaaatatggccaaaaacaagtgaagggaagtgaaaatccaagaagttattacaaatgcacataccccaattgccctacaaagaagaaggttgagaggtctttagatggacaaattactgagatagtttataagggtactcataaccatcctaagcctcaaaatactaggagaaactcatcaaactcctcttctcttgcaatccctcattcaaattccatcagaactgaaatcccagatcaatcctatgccacacatggaagtggacaaatggattcagctgccaccccagaaaactcatcaatatcaattggagatgatgattttgagcagagttcccaaaagtgtaaatcaggaggggatgaatatgatgaagatgaacctgatgccaaaagatggaaaattgaaggtgaaaatgagggtatgtcagcccctggaagtagaacagtgagagaacctagagttgtagttcagacaaccagtgacattgatatccttgatgatggctataggtggagaaaatacgggcagaaagtagtgaagggcaatccaaatccaaggagttactacaagtgcacacacccaggatgtccagtgaggaagcacgtggaaagagcctcacatgacctaagggctgtgatcacaacttatgagggaaagcacaaccatgatgttcctgcagcccgtggcagtggcagccattctgtgaacagaccaatgccaaacaatgcttcaaaccacaccaacactgcagccacttccgtaaggctcttgccagtgatccaccaaagtgacaattcccttcagaaccaaagatcacaagcaccaccagaagggcaatcacccttcaccctagagatgctacaaagtccaggaagttttggattctcagggtttgggaatccaatgcaatcttacgtgaaccagcagcaactatctgacaatgttttctcctccaggaccaaggaggagcctagagatgacatgttccttgagtctctactatgctgaaggaattttttttttccctttttggtagctatggaaggttggaaattttggaagtgggggactaggatttattggacaaataaggttccattcgatttattgcattttttggtttgttttgttgtaaattttatacagccacaggattggtatagtatatactagtatttcaaaaaaaaaaaaaaaaaa

SEQ ID NO: 10 - protein, 575 aa, Glycine max
MASSSGSLDTSASANSFTNFTFSTHPFMTTSFSDLLASPLDNNKPPQGGLSERTGSGVPKFKSTPPPSLPLSPPPISPSSYFAIPPGLSPAELLDSPVLLNSSNILPSPTTGAFVAQSFNWKSSSGGNQQIVKEEDKSFSNFSFQTRSGPPASSTATYQSSNVTVQTQQPWSFQEATKQDNFSSGKGMMKTENSSSMQSFSPEIASVQTNHSNGFQSDYGNYPPQSQTLSRRSDDGYNWRKYGQKQVKGSENPRSYYKCTYPNCPTKKKVERSLDGQITEIVYKGTHNHPKPQNTRRNSSNSSSLAIPHSNSIRTEIPDQSYATHGSGQMDSAATPENSSISIGDDDFEQSSQKCKSGGDEYDEDEPDAKRWKIEGENEGMSAPGSRTVREPRVVVQTTSDIDILDDGYRWRKYGQKVVKGNPNPRSYYKCTHPGCPVRKHVERASHDLRAVITTYEGKHNHDVPAARGSGSHSVNRPMPNNASNHTNTAATSVRLLPVIHQSDNSLQNQRSQAPPEGQSPFTLEMLQSPGSFGFSGFGNPMQSYVNQQQLSDNVFSSRTKEEPRDDMFLESLLC

SEQ ID NO: 11 - DNA, 2158 bp, Triticum aestivum
gcacgagccgcaccgcgccgatggccgattcgccaaaccctagctccggggacctcccctcagccgccgggagctcgcccgagaagccgtaccccgcggatcgacgcgtcgcggcgctcgccggcgcgggcgcgaggtacaaggccatgtccccggcgcggctgccgatctcgcgcgagccctgcctcaccatccccgccggcttcagcccctccgccctcctcgactcccccgtgctcctcaccaacttcaaggttgaaccttcaccaacaactggtagtctgagcatggctgcaattatgcacaagagtgctcatccagacatactgccttcgccacgggataagtctattcgagcccatgaagatgggggttctagggattttgaattcaagcctcatctgaattcgtcttctcaatcactggctcctgctatgagtgatctaaaaaaacacgagcattctatgcaaaatcagagtatgaatcccagctcatcatctagcaatatggtgaatgaaaacagacctccctgttcacgcgagtcaagtcttacagtgaatgtaagtgctccgaaccaacctgttggaatggttggtttgactgacaacatgcctgctgaagttggtacatctgagccgcagcagatgaatagttctgacaatgccatgcaagagccgcagtctgaaaatgttgctgacaagtcagcagatgatggctacaactggcgcaaatatgggcagaagcatgtcaagggaagtgaaaaccctagaagttattacaagtgcacacatcctaattgtgaagtaaaaaagctattggagcgtgcggttgatggtctgatcacggaagttgtctataaggggcgccataatcatcctaagccccagcctaataggaggttagctggtggtgcagttccttcgaaccagggtgaagaacgatatgatggtgcggcagctgctgatgataaatcttccaatgctcttagcaaccttgctaatccggtaaattcgcctggcatggttgagcctgttccagtttcagttagtgatgatgacatagatgctggaggtggaagaccctaccctggggatgatgctacagaggaggatttagagtcgaaacgcaggaaaatggagtctgcaggtattgatgctgctctgatgggtaaacctaaccgtgagccccgtgttgtcgttcagactgtaagtgaggttgacatcttggatgatgggtatcgttggcggaaatatggacagaaagttgtcaaaggaaaccccaatccacggagttactacaaatgcacaagcacaggatgccctgtgaggaagcatgttgagagagcatcgcacgatcctaaatcagtgataacaacgtatgaaggaaaacataaccatgaagtccctgctgcgaggaatgcaacccatgagatgtccgcgcctcccatgaagaatgtcgtgcatcagattaacagcagtatgcccagcagcattggcggcatgatgagagcatgtgaagccaggaacttcagcaaccaatattctcaagccgctgaaaccgacaatgtcagtcttgaccttggtgttgggatcagcccgaaccacagcgatgccacaaaccaaatgcagtcttcaggtcctgatcagatgcagtaccagatgcaatccatggcttcgatgtacggcaacatgagacatccatcatcaatggcagtgccaacggtacaaggaaactctgctggccgcatgtatggttccagagaagagaaaggtaacgaagggtttactttcagagccacaccgatggaccattcagctaacctatgctatagcggtgctgggaacttggtcatgggtccatgagaggaatgatgagagtgtcagcaaatgcttatagctccatgaatcatatattacaaacaatgcttttgtaacgacaatctcttcagcaagattcttaattgtgtatcggttacaagtcagttcagccagaggcaagtaagctataagctatacctggaggactgcagcaaatgcgcatgtgtctttttaggcgcggaaaaggcccctgctgtatgtagcgctgcagacctacattcgttgtacagcgaacctaatatgattaattaattagattatgagaatttggtttaaaaaaa

SEQ ID NO: 12 - protein, 619 aa, Triticum aestivum
TSRTAPMADSPNPSSGDLPSAAGSSPEKPYPADRRVAALAGAGARYKAMSPARLPISREPCLTIPAGFSPSALLDSPVLLTNFKVEPSPTTGSLSMAAIMHKSAHPDILPSPRDKSIRAHEDGGSRDFEFKPHLNSSSQSLAPAMSDLKKHEHSMQNQSMNPSSSSSNMVNENRPPCSRESSLTVNVSAPNQPVGMVGLTDNMPAEVGTSEPQQMNSSDNAMQEPQSENVADKSADDGYNWRKYGQKHVKGSENPRSYYKCTHPNCEVKKLLERAVDGLITEVVYKGRHNHPKPQPNRRLAGGAVPSNQGEERYDGAAAADDKSSNALSNLANPVNSPGMVEPVPVSVSDDDIDAGGGRPYPGDDATEEDLESKRRKMESAGIDAALMGKPNREPRVVVQTVSEVDILDDGYRWRKYGQKVVKGNPNPRSYYKCTSTGCPVRKHVERASHDPKSVITTYEGKHNHEVPAARNATHEMSAPPMKNVVHQINSSMPSSIGGMMRACEARNFSNQYSQAAETDNVSLDLGVGISPNHSDATNQMQSSGPDQMQYQMQSMASMYGNMRHPSSMAVPTVQGNSAGRMYGSREEKGNEGFTFRATPMDHSANLCYSGAGNLVMGP

SEQ ID NO: 13 - protein, 549 aa, Ipomoea batatas
MAASSGTIDAPTASSSFSFSTASSFMSSFTDLLASDAYSGGSVSRGLGDRIAERTGSGVPKFKSLPPPSLPLSSPAVSPSSYFAFPPGLSPSELLDSPVLLSSSNILPSPTTGTFPAQTFNWKNDSNASQEDVKQEEKGYPDFSFQTNSASMTLNYEDSKRKDELNSLQSLPPVTTSTQMSSQNNGGSYSEYNNQCCPPSQTLREQRRSDDGYNWRKYGQKQVKGSENPRSYYKCTHPNCPTKKKVERALDGQITEIVYKGAHNHPKPQSTRRSSSSTASSASTLAAQSYNAPASDVPDQSYWSNGNGQMDSVATPENSSISVGDDEFEQSSQKRESGGDEFDEDEPDAKRWKVENESEGVSAQGSRTVREPRVVVQTTSDIDILDDGYRWRKYGQKVVKGNPNPRSYYKCTSQGCPVRKHVERASHDIRSVITTYEGKHNHDVPAARGSGSHGLNRGANPNNNAAMAMAIRPSTMSLQSNYPIPIPSTRPMQQGEGQAPYEMLQGSGGFGYSGFGNPMNAYANQIQDNAFSRAKEEPRDDLFLDTLLA

SEQ ID NO: 14 - DNA, 36 bp, Artificial Sequence
  Description of Artificial SequenceSynthetic oligonucleotide
gaaaaattcatcagtggatccttatttgacctgtct